(19) United States
(12) Patent Application Publication
(10) Pub. No.: US 2024/0412871 A1
(43) Pub. Date: Dec. 12, 2024

(54) CLINICAL DECISION SUPPORT SYSTEM AND METHOD

(71) Applicant: Ohio State Innovation Foundation, Columbus, OH (US)

(72) Inventor: Puneet Mathur, Dublin, OH (US)

(21) Appl. No.: 18/739,226

(22) Filed: Jun. 10, 2024

Related U.S. Application Data
(60) Provisional application No. 63/507,213, filed on Jun. 9, 2023.

(51) Int. Cl. G16H 50/20; G16H 10/60; G16H 50/30; G16H 70/20

(52) U.S. Cl. CPC G16H 50/20; G16H 10/60; G16H 50/30; G16H 70/20

(57) ABSTRACT

A Clinical Decision Support System (CDSS) provides a comprehensive system to capture a patient's clinical encounter and observation data to inject into a risk calculation algorithm to align with alerts that can support physicians to make clinical decisions for treatment regimes. A software architecture and framework is created with functionality specific modules to develop a CDSS system for a disease area.

### RELATED APPLICATION

This U.S. application claims priority to, and the benefit of, U.S. Provisional Patent Application No. 63/507,213, filed Jun. 9, 2023, which is incorporated by reference herein in its entirety.

### STATEMENT OF GOVERNMENT SUPPORT

This invention was made with government support under Grant No. R01HL134673 awarded by the National Institutes of Health. The government has certain rights in the invention.

### BACKGROUND

Pulmonary arterial hypertension (PAH) is a chronic, rapidly progressive disease which is incurable. There are benefits to having an accurate risk-prediction tool that allows the determination of patients' prognoses, identifies treatment goals, helps patients make informed decisions, and monitors disease progression are needed. Risk prediction in PAH utilizes a range of parameters that must be performed periodically to plot individual patient trajectories and treatment interventions. Existing approaches for assessing risk in PAH patients include the use of equations and scores, developed from contemporary PAH registries.

However, these risk stratification tools vary in their precision, nature of their derivation, and utility for periodic use. They assume that the clinical variables that contribute to PAH risk are independent, linear in robustness, and limited to established variables. Their versatility is further limited by the fact that practitioners often rely on clinical ‘gestalt’ while managing patients, dismissing the available tools. Also, no adult based PH severity scores are customized/validated for pediatrics, leaving pediatric clinicians without guidance for patient counseling, appropriate drug treatment and clinical trial screening. Probabilistic risk-models derived from traditional statistical methods or expert opinion are insufficient for phenotyping complex diseases like PAH, as they fail to account for functional associations between parameters that may converge to an individual patient's risk.

Physicians' abilities to comprehensively assess patients with pulmonary artery hypertension (PAH), determine their prognosis, and monitor disease progression and response to treatment remains critical in optimizing outcomes. Accurate risk prediction remains essential to making individualized treatment decisions in PAH. Contemporary PAH risk stratification tools vary in precision, nature of derivation, applicability to varied subsets of PAH, extent of validation, utility for serial use, and the number of modifiable data elements. They are based on an outdated set of clinical variables, and neglect modern diagnostic tools that are now commonplace, such as new biomarkers, imaging and genomic fingerprints. Probabilistic models like REVEAL and the ERS/ECS scores are insufficient for phenotyping patients with complex cardiovascular pathology involved in PAH. They do not account for functional associations between diverse parameters that may converge to define patient subsets. Clinical profiles in the contemporary PAH population diverge widely from classical descriptions (e.g., plexogenic vascular remodeling & cor pulmonale) from which traditional risk variables were derived.

Clinical decision support systems enable integrated workflows, provide assistance at the time of care and offer care plan recommendations. A CDSS must integrate with a healthcare organization's clinical workflow, which is often already complex and made even more so by the integration. Some clinical decision support systems are standalone products that lack interoperability with reporting and EHR software, limiting their usefulness in clinical and administrative settings.

There is a benefit to improving clinical decision support systems.

### SUMMARY

An exemplary Clinical Decision Support System (CDSS) is described that provides a comprehensive system to capture a patient's clinical encounter and observation data to inject into a risk calculation algorithm to align with alerts that can support physicians to make clinical decisions for treatment regimes. A software architecture and framework is created with functionality-specific modules to develop a CDSS system that can calculate risk for a variety of disease areas.

The CDSS system can employ Bayesian statistical analysis and other machine learning analyses to evaluate disease, such as pulmonary arterial hypertension. The clinical decision support system and associated analysis can be used in a clinical workflow to provide individualized risk stratification analysis to facilitate complex decision-making processes in the treatment or diagnosis of the patient as well as for the design of clinical trials. In one example, the analysis has been validated and observed to have a receiver operating curve (ROC) of 0.81 for predicting one-year survival. The Bayesian statistical analysis and clinical decision support systems can additionally include seamless integration with clinical workflow and individualized risk stratification analysis to facilitate complex decision-making for both adults and pediatric PAH patients.

The clinical decision support system can provide system architecture, and enhanced prognostic models that include interactions with international imaging and pediatric registries and the FDA. A multi-center National “Risk” Meta registry may be generated using machine learning to map best practices. The clinical decision support system can be used to guide appropriate diagnostic work up, stratify risk, tailor individualized therapeutic decisions, and optimize the clinical trial design. The setup for an exemplary PHORA system may utilize an ongoing PAH registry (REVEAL) [8] and a subject-level data, harmonized Federal Drug Administration (FDA) database of completed clinical trials in PAH.

The PHORA system may be further layered with prospective, observational sessions with PAH physicians for 1) to the user interface (aka “front end”); 2) system architecture (aka “back end”); and 3) enhanced prognostic models, e.g., that include novel interactions with other NIH funded projects, international imaging and pediatric registries and the FDA.

In some aspects, the techniques described herein relate to a clinical decision support system including: a processor; a memory having instructions stored thereon; and a means for input and output, wherein at least one set of input variable data are provided by the input means, wherein execution of the instructions by the processor causes the processor to execute one or more risk algorithms, wherein each of the one or more risk algorithms is configured to generate a risk score value for a disease area associated with the risk algorithm using a subset of the input variable data.

In some aspects, the techniques described herein relate to a clinical decision support system including: a processor; a memory having instructions stored thereon; and a means for input and output, wherein at least one set of input variable data are provided by the input means, wherein execution of the instructions by the processor causes the processor to execute a risk algorithm configured to generate a risk score value for a disease area, and wherein the clinical decision support system is configured to display a set of risk score value (e.g., in a plotted line, the measured metrics of the patient) computed by the one or more risk algorithms associated with a first set of input variable data.

In some aspects, the techniques described herein relate to a clinical decision support system, wherein the clinical decision support system is configured to display a second risk score value (e.g., in the same plotted line, the predictive risk assessment) associated with a second set of input variable data or parameters with the displayed first risk score value.

In some aspects, the techniques described herein relate to a clinical decision support system, wherein the first and/or second risk score value is categorized into low risk (>95% chance of survival in 1 year), medium risk (% 95-%90 chance of survival in 1 year), high risk (<90% chance of survival in 1 year).

In some aspects, the techniques described herein relate to a clinical decision support system, wherein execution of the instructions by the processor causes the processor to query a lookup table of clinical treatment guidelines for the disease area.

In some aspects, the techniques described herein relate to a clinical decision support system, wherein the memory further includes a database for storing input variable data for one or more input instances.

In some aspects, the techniques described herein relate to a clinical decision support system, wherein execution of the instructions by the processor causes the processor to calculate the influence of the set of input variable data on the associated risk score value.

In some aspects, the techniques described herein relate to a clinical decision support system, wherein one of the risk algorithm includes an ensemble of one or more Bayesian (neural) networks, wherein the one or more Bayesian networks are tree-augmented Naive Bayes (TAN) networks.

In some aspects, the techniques described herein relate to a clinical decision support system, wherein the risk algorithm is one of a plurality of risk algorithms, each associated with a different disease area.

In some aspects, the techniques described herein relate to a clinical decision support system, wherein the disease area is Pulmonary Arterial Hypertension.

In some aspects, the techniques described herein relate to a method of operating a clinical decision support system for pulmonary hypertension, the method including: receiving, from a database, a first set of input variable data of a set of input variables; determining, via one or more pulmonary arterial hypertension risk algorithms, a first set of risk score values associated with a patient surviving within a given time period (e.g., wherein the given time period is within a month, within 3 months, within 6 months, or within 1 year) using the electronic medical records for a first set input variable data, for one or more time instances (e.g., current and past); outputting, via a visualization output of a graphical user interface associated with a user's device, the first set of risk score values associated with a patient surviving within the given time period; presenting, via the graphical user interface, a set of input variables for a second set of input variable data, wherein the second set of input variable data includes a portion or all of the set of input variables; receiving, from the user's device, the second set of input variable data provided by the user through the graphical user interface; determining, via the one or more pulmonary arterial hypertension risk algorithms, a second set of risk score values associated with the patient surviving within the given time period using the second set of input variable data; and outputting, via the visualization output of the graphical user interface, the second set of risk score values associated with a patient surviving within the given time period, wherein the second set of risk score values is concurrently presented with the first set of risk score values in the visualization output.

In some aspects, the techniques described herein relate to a method, wherein the visualization output is configured to (i) present a current risk score value of the first set of set of risk score values, including for a first time instance, (ii) present historical risk score values of the first set of risk score values, including at least for a second time instance and a third time instance, and (iii) present future risk score values of the second set of risk score values.

In some aspects, the techniques described herein relate to a method, further including: determining relative weights of each input variable of the set of input variables in determining the first set of risk score values associated with the patient surviving within the given time period; and outputting, via the graphical user interface, one of more indicators of determined relative weights of the candidate variable inputs (e.g., wherein the one or more indicators can be used by a physician to identify the candidate variable inputs of importance to focus treatment).

### BRIEF DESCRIPTION OF DRAWINGS

FIG. 1 illustrates an example implementation of a Clinical Decision Support System (CDSS) and the components thereof including the Pulmonary arterial hypertension (PAH) risk module.

FIG. 2 illustrates a schematic of the method of operating a clinical decision support system for pulmonary hypertension.

FIG. 3A illustrates an example implementation of a CDSS and the components thereof including the Pulmonary arterial hypertension (PAH) risk module.

FIG. 3B an example implementation of a CDSS and the components thereof including the PAH risk module.

FIG. 4 illustrates the structure of the Pulmonary Hypertension Outcomes Risk Assessment (PHORA) Bayesian network model, with conditional probability table for survival. PVR: pulmonary vascular resistance; NT-proBNP: N-terminal pro-BNP; BP: blood pressure; RAP: right atrial pressure; 6MWD: 6-min walk distance; NYHA: New York Heart Association; D_{LCO}: diffusing capacity of the lungs for carbon monoxide; WHO: World Health Organization.

FIG. 5 illustrates performance of the Bayesian networks algorithm when internally validated in the Registry to Evaluate Early and Long-Term PAH Disease Management (REVEAL); (PHORA area under the curve (AUC) 0.80), and externally in the Pulmonary Hypertension Society of Australia and New Zealand (PHSANZ; AUC 0.80) and Comparative Prospective Registry of Newly Initiated Therapies for Pulmonary Hypertension (COMPERA; AUC 0.74) registries.

FIG. 6 illustrates Kaplan-Meier curves demonstrating PHORA's risk-stratification abilities into low, intermediate, and high risk of 12-month mortality based on the 2015 European Society of Cardiology/European Respiratory Society guidelines in a) the REVEAL; b) the COMPERA; and c) PHSANZ registry.

FIG. 7 illustrates a) Example of a PHORA model when some variables (highlighted in blue) are observed at baseline assessment. The values of these variables are noted in the dotted line box adjacent to each node. Variables in orange are yet to be reported as patients are undergoing work-up. b) Updated PHORA model when additional parameters (previously in orange) are now available. Note change in the predicted outcome (survival at 12 months, green box) as additional data is input. PVR: pulmonary vascular resistance; eGFR: estimated glomerular filtration rate; NT-proBNP: N-terminal pro-BNP; BP: blood pressure; RAP: right atrial pressure; 6MWD: 6-min walk distance; NYHA: New York Heart Association; DLCO: diffusing capacity of the lung for carbon monoxide; WHO: World Health Organization; CTD: connective tissue disease.

FIG. 8 illustrates an example implementation of a CDSS and the components thereof including the PAH risk module.

FIG. 9 illustrates biomarker-based clusters in PAH Biobank. A) Distribution of biomarkers by cluster; B) Kaplan-Meier curve; and C) Forest plots of risk for death/transplant based on cluster membership.

FIG. 10 illustrates a neural network model based on learned pathways.

FIG. 11 illustrates a prototype ensemble model combining clinical and genetic models.

FIG. 12 illustrates PHORA-USE and Meta registry ecosystem.

FIG. 13 illustrates the PHORA-USE local registry, which provides clinicians with visual analytics of their local site population.

FIG. 14 illustrates a visualization of common treatment sequences of patients extracted from HER data.

FIG. 15 is an illustration an example implementation of a Clinical Decision Support System (CDSS) framework.

FIG. 16 is an illustration of an example implementation of a CDSS including a Pulmonary arterial hypertension (PAH) risk module; and

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

To facilitate an understanding of the principles and features of various embodiments of the present invention, they are explained hereinafter with reference to their implementation in illustrative embodiments.

In one aspect of the disclosure, an enhanced risk prediction algorithm is developed using machine learning, deep learning, and statistical methodology. In one aspect, the enhanced risk prediction algorithm is a Bayesian algorithm. In some embodiments, the Bayesian algorithm is an ensemble of Tree-augmented Naïve (TAN) Bayes algorithms. In some implementations, the algorithm integrated traditional clinical variables with new biomarkers as well as imaging and genomic data. Each class of variables (e.g. clinical, biomarkers, imaging, and genomic), is represented by a separate TAN model. Each TAN model is trained on a discrete set of variables; in some aspects, the variables are selected based on physician surveys, independent statistical analysis (e.g. Cox analysis), or other means for variable selection that are known in the field. The selected variables are related to measurable or discretized factors related to Pulmonary arterial hypertension. The ensemble of TAN models is further trained on the selected variables and provides a value of risk for survivability based on patient input variables.

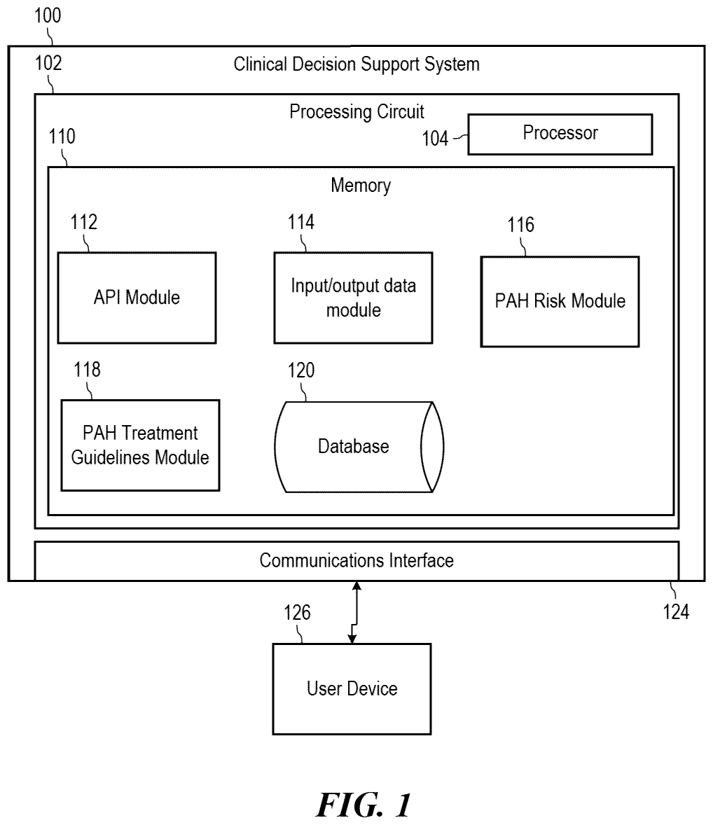

In another aspect of the disclosure, a clinical decision support system, hereafter CDSS, for clinicians of PAH patients includes the enhanced risk prediction algorithm, the PHORA model. An example CDSS is shown in FIG. 1, including an example computational system including a processing circuit 102, a communications interface 124 and practically coupled to a user device 126. The processing circuit 102 may include a processor 104 and memory 110. The processor 104 may be configured to execute instructions stored in the memory 110. In other examples, the instructions may be stored on a non-transitory computer-readable medium or on a cloud-based server. The memory 110 may have instructions stored thereon including a PAH Risk module 116, an API module, an input/output variable data module 114, a PAH treatment guidelines module 118, and a database 120.

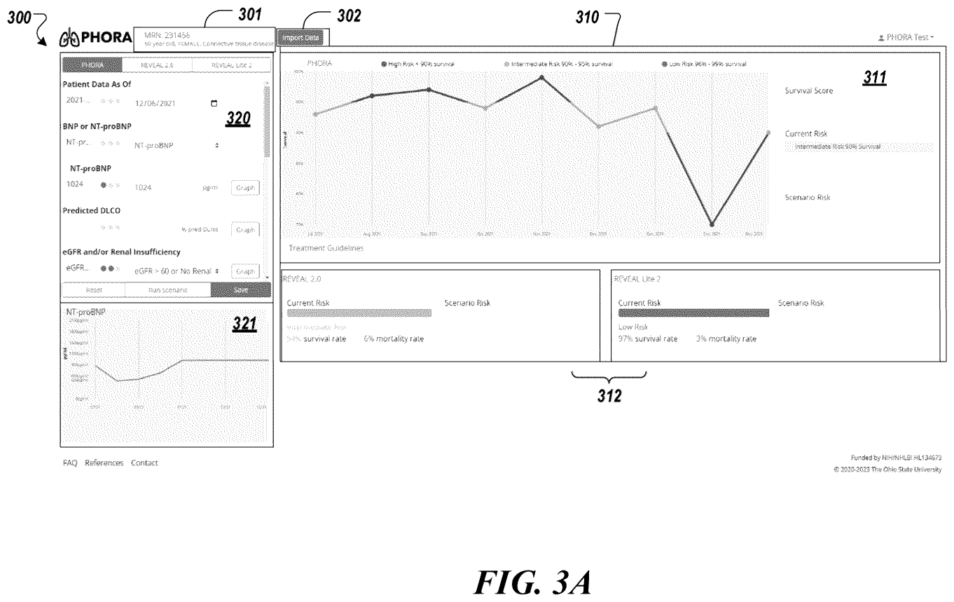

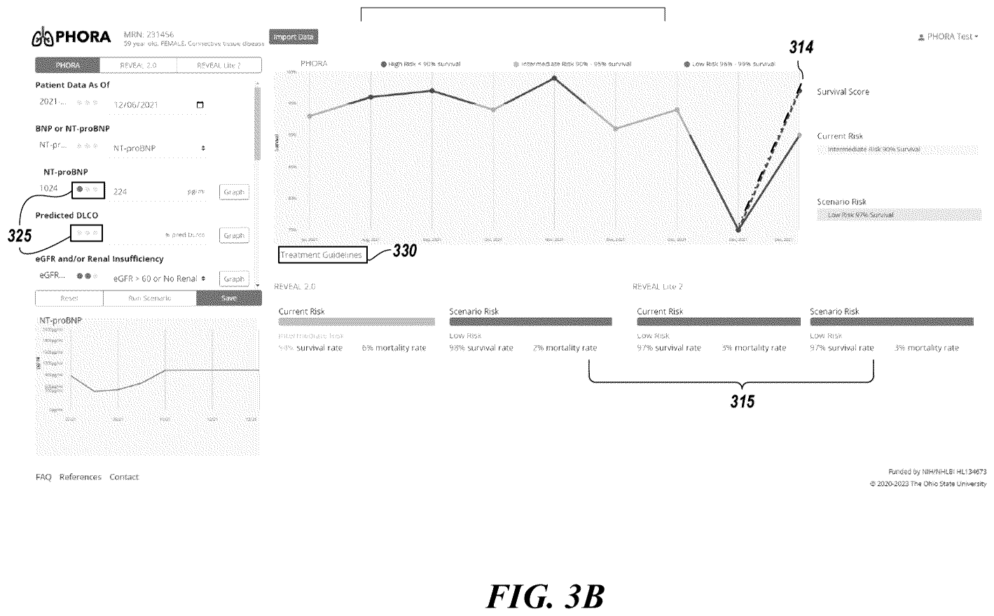

In some examples, the PAH Risk module 116 includes the PHORA model and other PAH risk prediction models. The PAH Risk module 116 may output the calculated risk of non-survival from the PHORA model together with other PAH risk prediction models for comparison for a patient. The PAH Risk module 116 may additionally provide output of calculated risk of non-survival over one or more time periods for the pateitn and output related trend lines per FIG. 3A.

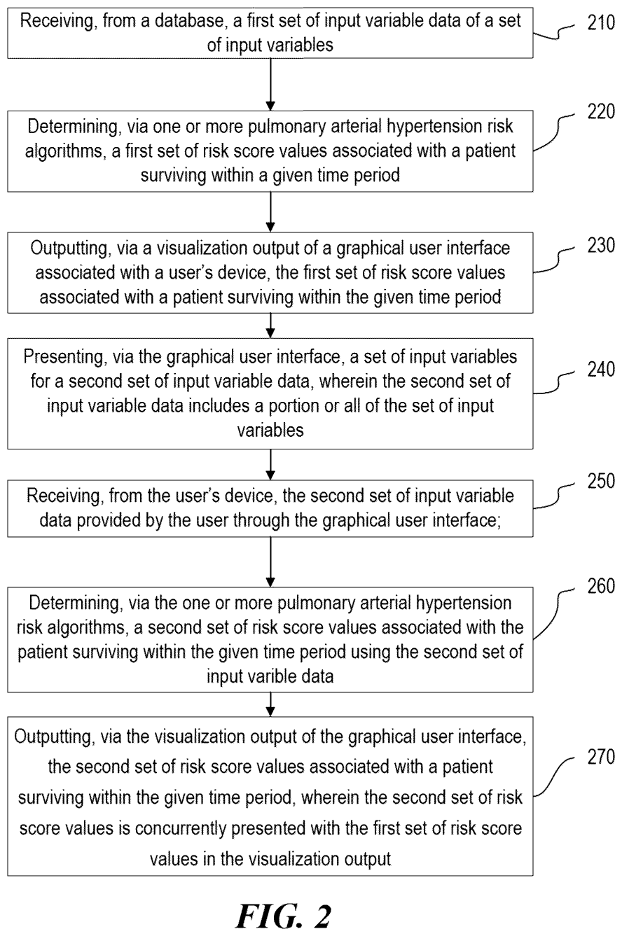

In other aspects, a method of operating the CDSS for pulmonary hypertension is described. As shown in FIG. 2, the method may include receiving 210, from a database, a first set of input variable data of a set of input variables; determining 220, via one or more pulmonary arterial hypertension risk algorithms, a first set of risk score values associated with a patient surviving within a given time period (e.g., wherein the given time period is within a month, within 3 months, within 6 months, or within 1 year) using the electronic medical records for a first set input variable data, for one or more time instances (e.g., current and past); outputting 230, via a visualization output of a graphical user interface associated with a user's device, the first set of risk score values associated with a patient surviving within the given time period; presenting 240, via the graphical user interface, a set of input variables for a second set of input variable data, wherein the second set of input variable data includes a portion or all of the set of input variables; receiving, from the user's device, the second set of input variable data provided by the user through the graphical user interface; determining 260, via the one or more pulmonary arterial hypertension risk algorithms, a second set of risk score values associated with the patient surviving within the given time period using the second set of input variable data; and outputting 270, via the visualization output of the graphical user interface, the second set of risk score values associated with a patient surviving within the given time period, wherein the second set of risk score values is concurrently presented with the first set of risk score values in the visualization output.

In some aspects, the visualization output of the method is configured to (i) present a current risk score value of the first set of set of risk score values, including for a first time instance, (ii) present historical risk score values of the first set of risk score values, including at least for a second time instance and a third time instance, and (iii) present future risk score values of the second set of risk score values.

In other aspects, the method further comprises determining relative weights of each input variable of the set of input variables in determining the first set of risk score values associated with the patient surviving within the given time period; and outputting, via the graphical user interface, one of more indicators of determined relative weights of the candidate variable inputs (e.g., wherein the one or more indicators can be used by a physician to identify the candidate variable inputs of importance to focus treatment).

In one aspect, the CDSS is a web application that shows output of the PAH risk prediction models in one or more visual modalities. As shown in FIG. 3A, the CDSS web application 300 provides a plurality of visual modalities including identification of the patient 301, a means for importing data through a GUI 302, risk stratification 310 of a selected PAH risk predication model 311, risk stratification of comparative PAH risk prediction models 312 selection of variables 320, and graphical representation of a selected variable 321.

The risk stratification visualization modality may show risk stratification of the selected PAH risk prediction model 311 at one or more time points for low risk, intermediate risk, and high risk. In some examples, the risk stratification output may be depicted by color or numerical means. The demarcation of risk stratifications may be commensurate with clinically recognized guidelines. For example, low risk may be ≥95% survival rate, intermediate risk 90%-95% survival rate, and high risk may be ≤90% survival rate.

In another aspect, the CDSS web application 300 provides a selection of variables 320 and may provide a means for variable manual input. In some aspects, the selection of variables 320 may include an option for graphically displaying the patient input variable values over time 321.

In other aspects, the CDSS may be used to run scenarios based on user-supplied inputs for the patient. For example, a user may change one or more of the patient's input variable values based on a planned course of treatment and request the CDSS to produce a second risk prediction output. A second risk prediction output may be displayed concurrently with a first risk prediction output 314. The second risk prediction may also be presented in the comparative PAH risk prediction models 315. In some aspects, the CDSS may provide output associated with the relative weights of the selection of variables 325. An example output display is shown in FIG. 3B. The combination of outputs—the relative weights of the selection variables 325 and second risk prediction output 314, 315 based on user supplied patient input variable values—provides the user targeted information to determine what patient variables to target to make the most impact in PAH risk outcomes. For example, the CDSS web application may display that the variable “eGFR” is the highest weighted variable of the selection variables for a patient and that the patient has a currently high risk stratification. The user may run a scenario with an different “eGFR” patient input variable value than currently measured, which results in the CDSS providing a second rick prediction output for the user. In this example, a change in the “eGFR” variable value may change the risk evaluation from high risk to low risk, with a higher chance of survival.

In other aspects, the CDSS may include a PAH Treatment guidelines module 118, and in the CDSS web application 300 may provide suggested treatment guidelines 330 based on the current risk stratification. The treatment guidelines may be looked up from a clinically accepted set of guidelines for treatment of PAH.

### EXAMPLES

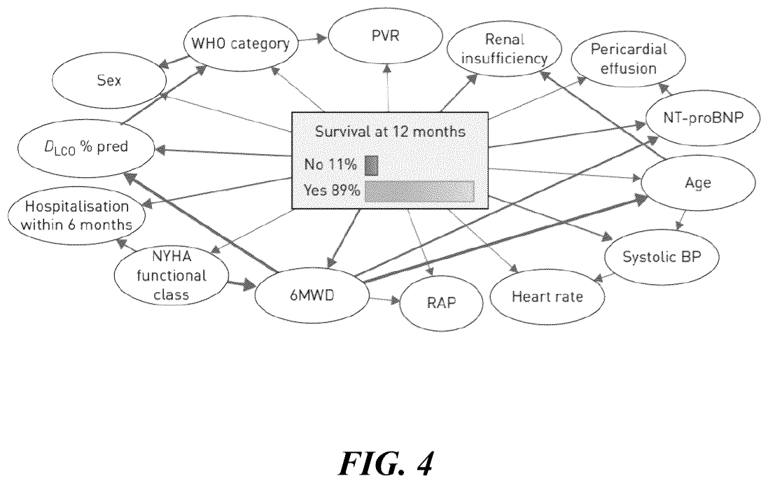

Example #1—Enhanced risk prediction algorithm (PHORA): Bayesian networks incorporate relationships and processes in individual patient data within a large dataset to predict probability of the outcomes for survival and adverse events. Tree-augmented Naïve (TAN) Bayes algorithms for structure and parameter learning were used for a Pulmonary Hypertension Outcomes Risk Assessment model, hereafter the PHORA model [59, 60]. TAN architecture adds a level of complexity to the simplest network form (a naïve Bayes), allowing independent variables to both directly and indirectly impact the outcome through their influence on other variables. These inferences are represented diagrammatically (FIG. 4), in which nodes represent pertinent variables and directed arrows between nodes represent interactions between those variables. Absence of an arrow between a pair of nodes implies independence between those variables. Only patients who had data at the 1-year mark available were included, using variables at 12 months, if available. If there was no assessment done at 1 year, the variable most recent to that time point (including assessment at enrolment, up to 12 months) was used. The TAN model was structured from the database, variables and cut-points shown in Table 1, looking at survival at 12 months as the clinical outcome. Clinical variables were coded as nodes, which were then discretised into prespecified intervals (e.g. N-terminal pro-brain natriuretic peptide levels (<300, 300-1100, >1100 pg·mL⁻¹) or 6-min walk distance (<165, 165-320, 320-440, >440 m)), as required for Bayesian methodology. The Bayesian network model learned the direction and magnitude of influence between these prespecified variables on each other as well as the final clinical outcome, represented in the model as conditional probability tables. The final model represents the joint probability distribution over its variables, by taking the product of all prior and conditional probability distributions (FIG. 4). The PHORA model used GeNIe software developed at the University of Pittsburgh, although any other suitable artificial intelligence software platform may be used. GeNIe is a machine-learning software which provides a platform for artificial intelligence modelling based on Bayesian networks.

TABLE 1
List of variables and their discrete states from the Registry to Evaluate
Early and Long-Term PAH Disease Management (REVEAL 2.0) risk score.

| | | Nodes in |
|---|---|---|
| Risk Factor in REVEAL | Random variable | Bayesian network |
| CTD-PAH Heritable POPH | WHO group I | CTD |
| | | Heritable |
| | | POPH |
| | | Other |
| Male >60 year | Sex | Female |
| | | Male |
| | Age years | ≤60 |
| | | >60 |
| Comorbidity | eGFR <60 ml · min⁻¹ · 1.73 m⁻² or renal | Yes |
| | insufficiency if eGFR is unavailable | No |
| eGFR <60 ml · min⁻¹ · 1.73 m⁻² or renal | | |
| insufficiency if eGFR is unavailable | | |
| NYHA functional class I | NYHA/WHO functional class | I |
| NYHA functional class III | | II |
| NYHA functional class IV | | III |
| | | IV |
| Systolic blood pressure <110 mmHg | Systolic blood pressure mmHg | <110 |
| | | ≥110 |
| Heart rate >96 bpm | Heart rate bpm | ≤96 |
| | | >96 |
| 6MWD <165 m | 6MWD m | <165 |
| 6MWD 320-440 m | | 165 to <320 |
| 6MWD ≥440 m | | 320-440 |
| | | ≥440 |
| BNP <50 pg · mL⁻¹ or NT- | BNP or NT-proBNP pg · mL⁻¹ | <50 or <300 |
| proBNP <300 pg · mL⁻¹ | | 50-200 or 300-1100 |
| BNP 200 to <800 pg · mL⁻¹ | | 1100 |
| BNP ≥800 pg · mL⁻¹ or NT- | | 200-800 |
| proBNP ≥1100 pg · mL⁻¹ | | ≥800 or ≥1100 |
| Echocardiogram Pericardial effusion | Pericardial effusion | Yes |
| | | No |
| Right heart catheterization | Mean RAP | <20 |
| Mean RAP ≥20 mmHg | | ≥20 |
| within 1 year | | |
| PVR <5 WU | PVR WU | <5 |
| | | ≥5 |
| PFT D_{LCO} <40% | % D_{LCO} | <40 |
| | | ≥40 |
| Hospitalization in 6 months | Hospitalization in 6 months | Yes |
| | | No |
| Survival at 12 months | Survival at 12 months | Yes |
| | | No |

Patient population/validation cohorts: The PHORA Bayesian network model was validated both internally and externally, utilizing the following cohorts and methodologies. The PHORA model was validated internally within the REVEAL registry using 10-fold cross-validation and the results of this validation were reported as AUC. While the PHORA model was validated externally in two registries: 1) the COMPERA registry, which is an ongoing multinational European registry comprised of patients with pulmonary hypertension/PAH enrolled since May 2007 [5]. The PHORA model was validated on 3849 newly diagnosed, consecutively enrolled PAH patients. Data from time of enrolment were considered; 2) the Pulmonary Hypertension Society of Australia and New Zealand (PHSANZ) Registry, which collects data from patients with all subgroups of pulmonary hypertension since December 2011 from 16 Australian and two New Zealand centres [61]. PHORA was validated in those PAH patients who had 1-year data available (978 out of 1076). Variables included were at the time closest to 1-year mark, as available. These included both previously (75%) and newly diagnosed (25%) PAH patients within the PHSANZ registry.

PHORA performance in predicting survival in each registry was measured using the AUC method. Kaplan-Meier curves were then derived for the PHORA-predicted mortality risk thresholds (i.e., low risk<5% 12-month mortality; intermediate risk 5-10% 12-month mortality; high risk>10% 12-month mortality) based on the 2015 ESC/ERS guidelines [5]. The statistical significance of the ability of PHORA to stratify risk groups in each of the three registry populations was calculated using Chi-squared analysis.

Results: Of the 3515 patients enrolled in REVEAL, 2529 were in the registry at 12 months after enrollment and included in the PHORA model. Of these, 73.7% were previously diagnosed (i.e., >3 months before enrolment) and 26.3% were newly diagnosed (i.e., ≤3 months before enrolment). The majority of the patients were female (80%), New York Heart Association/World Health Organization functional class II (41.3%) or III (45.9%), with a mean age of 53.6 years.

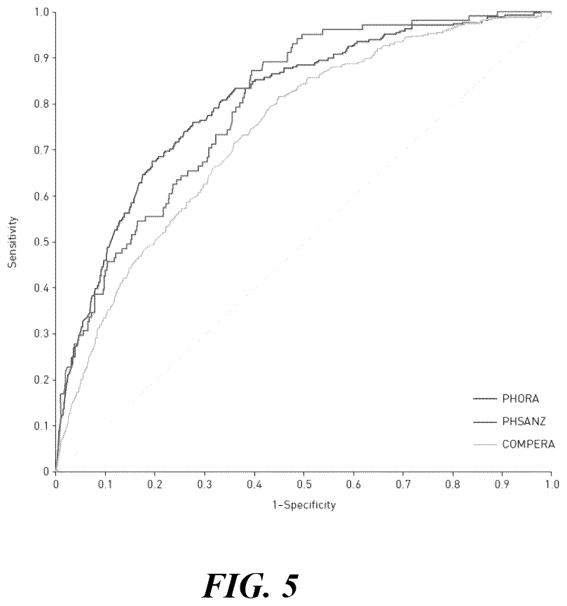

The AUC of 0.80 for predicting 1-year survival for the PHORA model indicated improved discrimination in predicting mortality over REVEAL 2.0 (0.76, 95% CI 0.74-0.78) and REVEAL 1.0 (0.71, 95% CI 0.68-0.77). PHORA had specificity of 0.76 (95% CI 0.69-0.84), sensitivity of 0.79 (95% CI 0.72-0.82), negative predictive value of 0.30 (95% CI 0.25-0.34) and positive predictive value of 0.97 (95% CI 0.96-0.98) for 1-year survival. PHORA demonstrated an AUC of 0.74 and 0.80 when validated in the COMPERA and PHSANZ registries, respectively (FIG. 5). Hence, PHORA outperformed the contemporary REVEAL 2.0 risk stratification model.

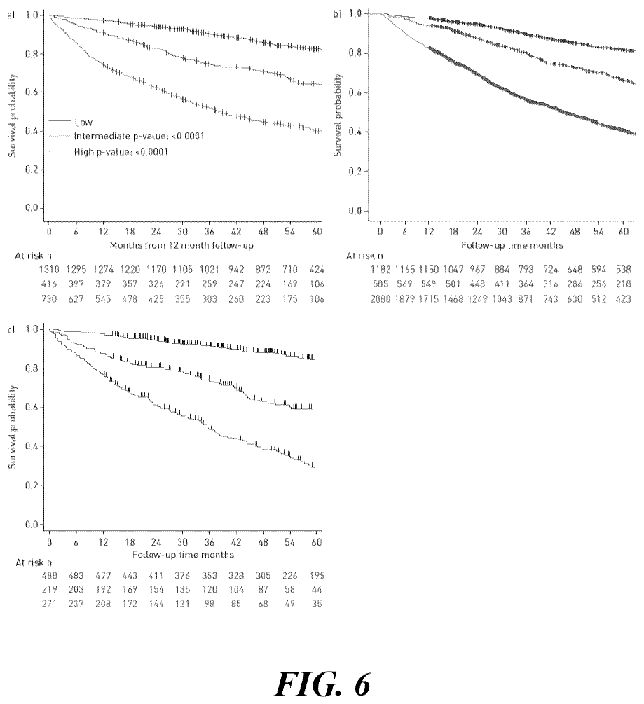

Patients were classified as low risk (<5% 12-month mortality); intermediate risk (5-10% 12-month mortality) and high risk (>10% 12-month mortality) based on the 2015 ESC/ERS guidelines. 12-month survival rates predicted by PHORA were greater for patients with lower risk scores and poorer for those with higher risk scores (p<0.001), with excellent separation between low-, intermediate- and high-risk groups in all three registries (FIG. 6). This demonstrates PHORA's ability to risk-stratify patients effectively early in the course of the disease, which would allow for appropriate clinical decision making.

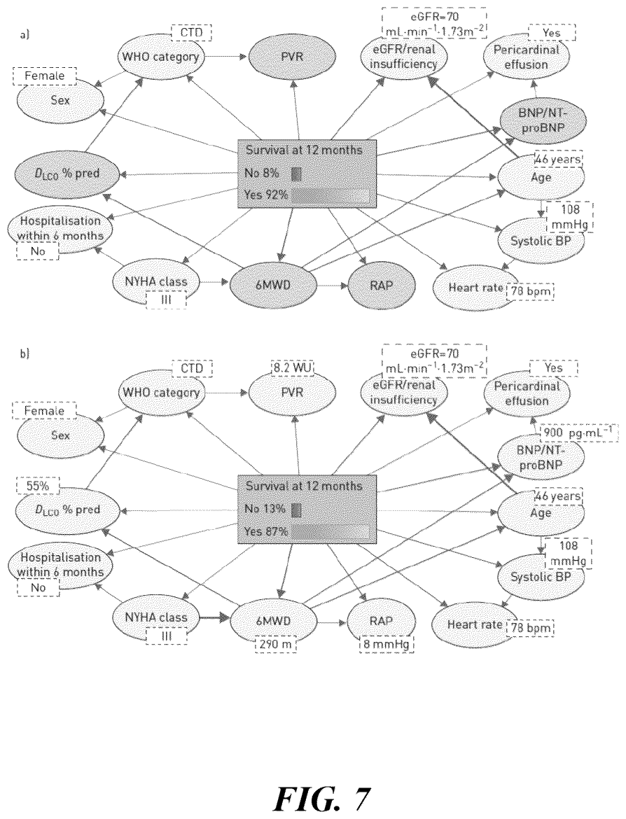

FIG. 7 demonstrates the ability of PHORA to illustrate the dynamic interdependencies among the variables. FIG. 7a) demonstrates the baseline probability relationships between variables in the model and the outcome during a baseline assessment of an example patient. FIG. 7b) shows how these baseline probability relationships of the network change with the addition of new variables as patient undergoes ongoing work-up.

Discussion: Risk stratification using the PHORA model, a Bayesian network model, provides improved discrimination to the existing Cox regression multivariate model and effectively depicted risk in two large external registry cohorts, COMPERA and PHSANZ. This improvement stems from the ability of the Bayesian network model to understand both the dynamic influences of each risk factor on each other, as well as with the outcome itself.

The utility of the Bayesian network methodology was only recognised within the past 25 years, with the publication and application of Bayesian network-based decision support tools in a variety of medical disciplines [62-65]. In these clinical scenarios, Bayesian network-based tools were noted to have superior predictive performance over traditional statistical methods [59]. Bayesian networks do not require restrictive modelling assumptions outside of expressing independencies whenever these are justified. Descriptively, Bayesian networks provide the advantages of a rigorous probabilistic framework that uses inference of multiple variables and a visual representation that is interactive and easy to interpret. This also allows a user to input these various scenarios and calculate the changes in predicted mortality and other adverse events in a highly interactive fashion. When performing prediction, Bayesian networks allow for estimating the outcome probability based on partial observations, as often happens in a clinical setting. Lastly, Bayesian networks offer more flexibility, such as allowing for missing values, and result in more intuitive models.

Appropriate risk-stratification tools are necessary to guide clinical treatment goals and monitor disease progression. Clinically, a good risk assessment tool should be evidence based, easy to administer, externally validated, have good discrimination (C-index>0.7), account for “missingness” in data, incorporate weighting of individual variables and reflect the dynamic interactions between variables as well the primary outcome [2]. In the development of contemporary risk stratification in PAH, investigators are limited in their ability to produce robust and highly discriminatory (i.e. C-index>0.8) predictive tools. This relates in part to reliance on registry datasets, which are limited in data quality, quantity and comprehensiveness. Although real-world in nature, these registries provide limited yield of high-quality data considering the differences in patient characteristics enrolled, number of patients observed, quality of data collected and failure to capture relevant variables (i.e., imaging or novel biomarkers) that could add substantially to the comprehensiveness and discriminatory power of equations and calculator. Another significant limitation to the predictive power of contemporary risk assessments is their reliance on traditional statistical methods (Cox proportional hazard) or expert opinion. Cox proportional hazard models allow for estimating the effect of multiple risk factors on survival, with the impact of each individual risk factor expressed by their hazard ratio. However, a hazard ratio remains constant over time and is unaffected by concomitant risk factors [66]. In addition, clinically relevant variables such as rate of disease progression remain unaccounted for [67]. Lastly, traditional models are not capable of handling several missing clinical variables, which may not have been obtained at the time of evaluation. This results in a unidimensional and sometimes oversimplified risk prediction, which lacks in robustness with respect to predicting outcome in complex disease. Thus, with limited datasets, the use of the described PHORA model, a set of Bayesian networks, could help with several of these shortcomings.

As per the 2015 ESC/ERS treatment guidelines, PAH should be risk-stratified as low (<5%), intermediate (5-10%) or high (>10%) risk of mortality at 12 months to enable guidance on therapeutic decisions. However, in clinical practice, some patients may present with a combination of low-, intermediate- or high-risk features, which can then cloud clinical judgment and misguide subsequent medical therapy. PHORA can be deployed as a decision tool in the clinical arena to integrate the sometimes conflicting information. Another unique advantage of PHORA is that it allows for estimation of the outcome probability based on partial observations, without knowledge of presence or absence of remaining risk factors (FIG. 7).

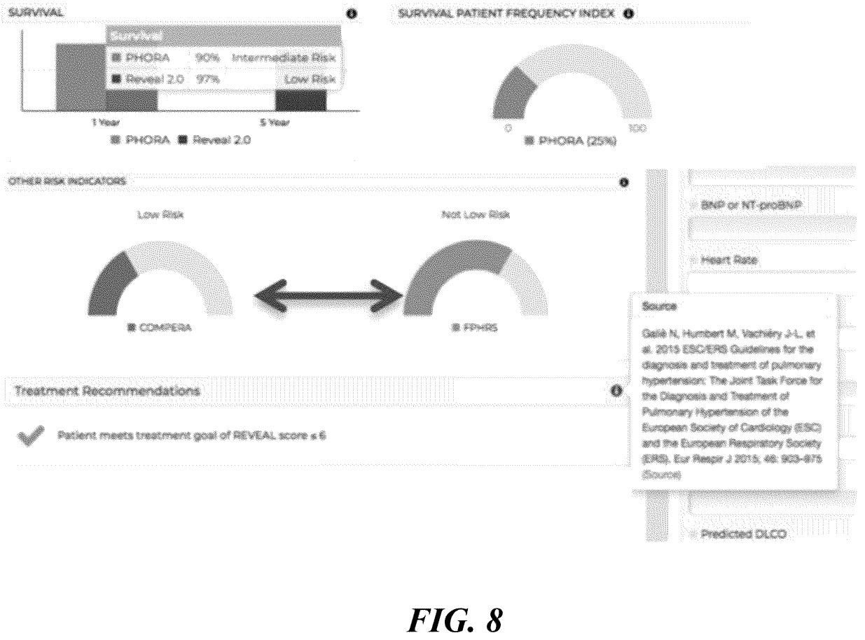

Although PHORA was derived from a prevalent patient registry (REVEAL), it was able to predict outcomes with equally good discrimination across two completely different real-world registries, regardless of whether patients were mostly incident (COMEPRA) or prevalent (PHSANZ). Lastly, longitudinal monitoring with PHORA could guide treatment strategies by providing a specific, quantitative metric for satisfactory clinical response (a relative reduction of baseline percentage risk as opposed to lowering a risk stratum). It is envisioned that PHORA outputs and clinical variable entry will be depicted in an easy-to-visualise format on a web-based application, along with comparative REVEAL 2.0, COMPERA and French scores [6, 58] (FIG. 8), allowing a side-by-side decision tool for clinicians to understand both the ranges in risk, the degree of influence of each variable on predicted outcome and likelihood scenario of each clinical case added.

It is contemplated that the derivation of the PHORA model from clinical registry data, including missing data pertaining to the independent variables, results in the loss of some robustness of risk predictions. The REVEAL database is large and representative, like other registries it suffers from incomplete capture of many data elements. This could impact the analysis by allowing patients used in both the model training and validation whom have up to 40% of their data missing. This could be particularly pertinent, if the missing data are related to the health of the patient per se (e.g. patient was too sick, so tests could not be done), thus skewing the analysis toward healthy patients. However, the fact that the model is not built on ideal complete' datasets and can handle data missingness is also reflective of real-life clinical scenarios where all clinical data may not be available at each time-point. An additional limitation is the dependency on REVEAL-based cut-points and data used to derive PHORA only reflected prevalent patients who were alive and in the study at 12 months of follow-up. This was done to account for all-cause hospitalization data in the previous 6 months, but raises concerns that the risk score is subject to survival bias. However, risk prognostication is typically not subject to survivor bias because risk is assessed only during the time the patient has participated in the registry. Whether a change in projected risk prediction scores in PAH reflects a true change in a patient's outcome remains a topic of debate. Lastly, interactions noted between the variables and survival are clinically likely to be even more complex than was captured by the TAN model.

In order to address these limitations, further derivation and validation studies using Bayesian networks that can appropriately handle mixed (categorical and continuous) data are additionally provided using a harmonised, contemporary clinical trial dataset (n>3000) in conjunction with the United States Food and Drug Administration (FDA). A combination of both feature engineering (evidence-based, expert guided selection), feature learning (via information scoring) and dimensionality reduction (via unsupervised methods) are incorporated in additional embodiments of the PHORA model with a key goal of maximising its discrimination (C-index>0.8), while keeping the tool easy to use. In other embodiments of the PHORA model, datasets will include REVEAL variables and other novel and significant variables determined by unsupervised modelling methods and further enhanced by expert opinion. Lastly, Bayesian network-based models at follow-up time-points can be evaluated to capture the impact of variables that may change over time allowing a more comprehensive prediction based on disease progression.

The FDA advocates the prospective use of patient characteristic(s) to select a study population in which detection of a drug effect (benefit, or lack thereof) is more likely than in an unselected population. The use of enhanced risk scores in PAH drug efficacy trials could accommodate enrolment of patients that are deemed to be at intermediate- or high-risk for clinical worsening, hence allowing for substantially smaller sample size and cost-saving.

The Bayesian network-derived risk prediction model, PHORA, demonstrated an improvement in discrimination over existing models. Bayesian network models have the advantage to learn from available data, incorporate expert knowledge, account for the interrelationships between clinical variables on outcome, and are more tolerant to missing data elements when calculating predictions. Hence machine learning based risk modelling can provide PAH clinicians with a greater level of confidence for making medical decisions in this complex, progressive disease.

State of the art prediction models fail to represent contemporary paradigms of PAH pathobiology [64]. The disclosed PHORA clinical decision support system (CDSS) was configured to include the biomarkers (ST-2, GDF-15, NT-ProBNP), imaging parameters (ECHO cardiography and cardiac MRI), and genomic variants and pathways. These enhancements were derived from clinical trials, including a robust subject level, harmonized dataset developed in conjunction with the Food and Drug Administration of the United State government (FDA), international and national registry collaboratives, as well as harmonized genomic dataset from the instant study and the PH National Biobank.

In addition to the improvements in accuracy, versatility, and robustness, the disclosed CDSS platform provides added capabilities used for future clinical trial enrichment and endpoint development.

Example #2—PHORA: Testing the Bayesian Approach [8, 18-21]: A first implementation of the Pulmonary Hypertension Outcomes Risa Assessment (PHORA 1.0) was developed using Tree Augmented Naïve (TAN) Bayes model to predict one-year survival in PAH patients included in the REVEAL registry (n=2,529), using the same variables and cut-points found in REVEAL 2.0 (22). The TAN architecture allowed independent variables to both directly and indirectly impact the outcome through their influence on other variables as shown in FIG. 4.

The first implementation of PHORA was validated internally in REVEAL registry and 10-fold cross validation and externally in COMPERA [5] and PHSANZ registry [23]. Patients were classified as low, intermediate and high-risk based on the 2015 ESC/ERS guidelines. The first implementation of PHORA had a Receiver operating curve (ROC) of 0.81 for predicting one-year survival, which was an improvement over REVEAL 2.0 (ROC of 0.76 When validated in COMPERA and PHSANZ registries, PHORA demonstrated a ROC of 0.74 and 0.80. There was an excellent separation between low, intermediate, & high-risk groups in REVEAL, COMPERA & PHSANZ (P<0.001). Two unique advantages of PHORA are the ability to illustrate the dynamic interdependencies among the variables (FIG. 4) & the ability to estimate outcome probability based on observations (i.e., allowing for missing data), without knowledge of the presence or absence of remaining risk factors.

PHORA CDSS for clinical use: The PHORA CDSS Web Applications employed the PHORA 1.0 Bayesian model for 1-year mortality, REVEAL 2.0 for 1- and 5-year mortality, the COMPERA model and the French Non-invasive Risk Score for low or high-risk stratification as shown in FIG. 8.

In some examples, the display of the PHORA CDSS Web Applications can indicate or show the mortality predictions with bar graphs and the European risk stratification methods with gauges. The blue gauge may represent the Bayesian model “patient frequency index,” a measure of the rarity of the patient given the information provided to the model per FIG. 8. This visual is designed to help interpret the confidence of the model (PHORA) as described herein. An additional feature of this example of the PHORA CDSS is an expert knowledge base derived from the PAH ESC/ERS guidelines [29]. Organization of information from these guidelines into a logical-dependency lookup table may be provided, e.g., with the table functionalized on the PHORA CDSS clinician application.

In the example shown in FIG. 8, patient data is shown that did not reach the treatment goal of attaining low-risk status. The clinician is alerted to this status and can see where these treatment recommendations arise from by hitting the “i” bubble. Further enhancements may be included to better visualize the Bayesian model, as shown in FIGS. 2 and 3).

Example #3—A second implementation of PHORA (PHORA 2.0): Feature Selection: Clinical trial data (Bayer, Actelion, and United Therapeutics) may be assessed for the relationships between different categories of variables (laboratory values, hemodynamics, functional capacity and demographics, and imaging) in relation to clinical outcomes (e.g., mortality, clinical worsening, and PAH-associated hospitalization). In conjunction with these statistics, univariate Cox's proportional hazard models may be conducted in their selected clinical trials to identify features for the PHORA model prediction.

In a study, feature learning was conducted in each data set using the significance of the Cox proportional hazards. Once completed, the study aggregated p-values of all datasets via meta-analysis (Stouffer method). Baseline hemodynamics and outcome were assessed in 2500 patients and represent a sufficiently large hemodynamic evaluation in PAH. These types of analyses were completed for laboratories, EKG, etc., from baseline and after 12-16 weeks to time to outcome.

Model Development: Once features were identified as predictors, they were assessed in data training dataset, a newly developed, harmonized dataset with the FDA using subject-level data. This harmonized dataset comprised seven clinical trials conducted from 2004-2019 with N=4300 individual patient-level data (PHIRST, AMBITION, PATENT, GRIPHON, FREEDOM-EV, SERAPHIN, and ARIES), and was used as the main dataset for Bayesian structure learning and initial parameter estimation for PHORA 2.0. Forty-one clinical variables were initially considered based on their p-value ranking from previous meta-analyses, availability across trials, and expert opinion.

A correlation heatmap was used to remove variables with moderate-to-strong correlation (R>0.6), with priority given to the most significant variables in the meta-analysis. Training data was created by random sampling of 80% of the harmonized dataset, dropping early censored patients (N=2531), leaving 20% of the data as a validation set (N=626). Continuous variables were discretized through univariate supervised decision trees using 10-fold cross-validation that maximized the Brier score.

A “genetic” search optimization was developed to determine candidate groups of features that maximized ranked correlation with the outcome (Kendall's tau for one-year survival) and reduced redundant features, with an increasing penalty for redundancy. This method established four feature combinations that were evaluated in Augmented Naïve Bayesian Network classifiers, and the best model was selected by multiple rounds of 10-fold cross-validation on training data. The final performance is reported as performance on the validation set.

The best final model, as determined from the genetic search of feature combinations, maintained high cross-validation (Average AUC 0.82) (FIG. 4). The final performance on the test set using this model was an AUC=0.85. The final model outperformed all other published risk calculators (including COMPERA, FPHN or French score, REVEAL 2.0 & PHORA 1.0) for predicting mortality at 1-year. Considering these results, the described Bayesian network modeling demonstrates compelling performance improvements upon the published performance of traditional PAH risk calculators. It is contemplated that further studies may optimize the described model performance and validate the model on real-world registry data.

Biomarker: The PHORA system and algorithms were integrated with both biomarker and genomic markers of risk from PH Biobank.

Exploratory evaluations of novel biomarkers (ST2, NT-proBNP, endostatin, HDGF, Gal3, IL6) have been conducted including measurements using a custom printed multiplex electrochemiluminescence based ELISA and clinical data were obtained from 2,017 adults and 182 children with Group I PAH from the PH Biobank. In adults, higher ST2 and NTproBNP levels were associated with an increased risk of death (hazard ratios 2.79, 95% CI 2.21-3.53, p<0.001 and 1.84, 95% CI 1.62-2.10, p<0.001 respectively) [31]. In multivariable modeling, serum IL-6 (32) was associated with survival in the overall cohort (hazard ratio 1.22, 95% CI 1.08-1.38; p<0.01). ST2 significantly improved the model (HR 2.05) over REVEAL 2.0 score (HR 1.88). In the adjusted analysis using pediatric PAHBiobank samples, while REVEAL 2.0 score was predictive of clinical worsening, the addition of ST2 significantly appears to improve the model (HR 2.05).

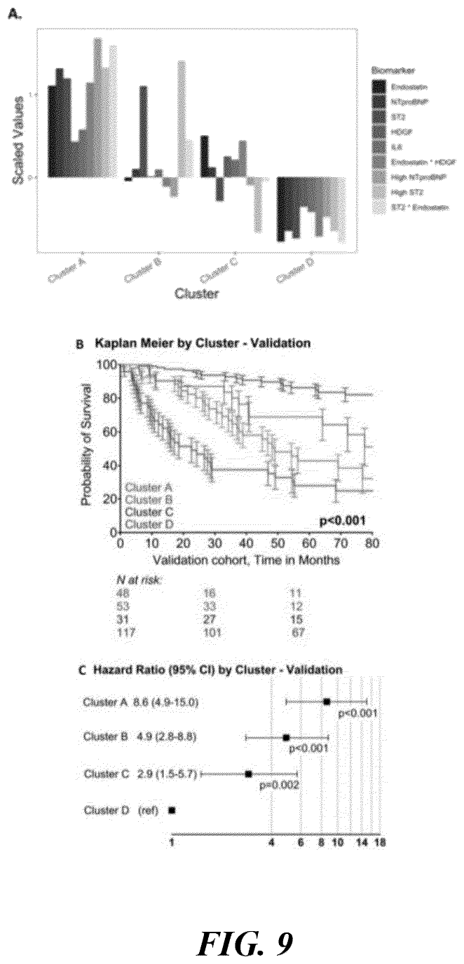

FIG. 9 shows preliminary analyses of an artificial intelligence unbiased cluster analysis to examine plasma biomarkers alone for survival risk using the PAHBiobank samples and clinical data. In one example, as shown in FIG. 9B, biomarker data alone produced four clusters and these clusters reflected a spectrum of mild to severe survival risks. It is contemplated that the addition of blood biomarkers to the PHORA model could improve severity/survival prediction.

A preliminary Genomic-wide association study (GWAS) study was conducted, which included novel biomarker discovery, together with agreements from the United Kingdom Assessing the Spectrum of PH Identified at Referral Center (ASPIRE) MRI database [35, 36], the Australian/New Zealand National ECHO database (NEDA) [37, 38], an innovative ECHO retrieval program with the PAH Biobank, and the US MRI registry.

In this example, a meta-analysis of GWAS results between the PH Biobank (n=1,885) and a prior study (n=911) was conducted. After adjustment for age, sex, prostacyclin use, and PVR, self-reported Hispanic patients exhibit significantly improved survival versus NHWs (p=0.009) (34). The evaluations were extended to determine independent genetic determinants of survival. GWAS data from AHN and PH Biobank (PAHB) were processed and cleaned at Indiana University using GWASTools based pipeline. Logistic regression was used with survival outcome as a dichotomized outcome variable. GWAS of the outcome was conducted separately in AHN and PAHB, then a meta-analysis of the two cohort was conducted. One survival loci (NCKAPL1, p-value<5×10₋₈) was identified that represents a potential target for validation. It is contemplated that once validated, this locus will be used to stratify risk PAH patients with as another implementation of PHORA.

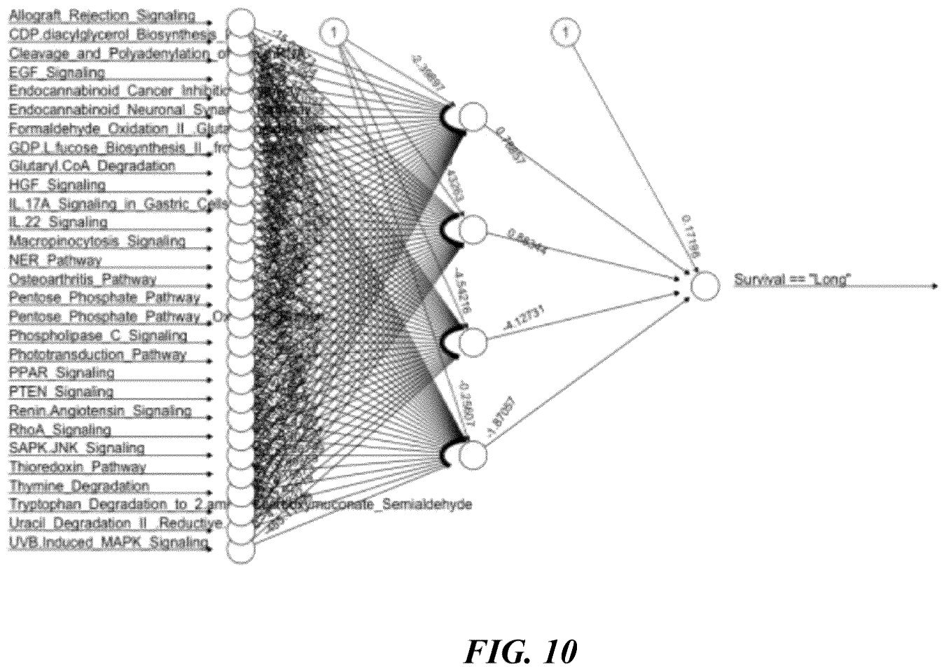

Whole genome sequencing has been performed on stored samples from 221 PAH patients. Samples were included with long survival greater than 7 years and short survival (<5 years). Variants were filtered for quality, assigned to genes, and filtered for function and population frequency. Genes are grouped based on Canonical Pathways defined in Ingenuity Pathway Analysis. Of pathways containing more than one gene mutated in three or more samples, twenty-nine were associated with survival length. Biologically relevant pathways include Pentose Phosphate (p=0.005), IL-22 (p=0.006), Phospholipase C signaling (p=0.007), Endocannabinoid related pathways (p=0.01), and Thioredoxin pathway (p=0.015). A Neural network model based on the top pathways was constructed per FIG. 10, which predicted long/short survival.

The PHORA algorithm including the TAN framework was configured to be able to discover & embed novel molecular biomarkers, genomics, imaging and clinical measurements.

Clinical data: Another study performed feature selections and subsequent model training for a third implementation of PHORA, PHORA 3.0, using modern machine learning methods. The FDA advocates the use of prognostic enrichment of clinical trials by preselecting a patient population with increased likelihood of experiencing the trial's primary endpoint. Validated clinical scales of risk (COMPERA, French score, REVEAL 2.0 and PHORA 1.0) were compared to identify patients that are likely to experience a clinical worsening event for a trial [25, 26]. Power simulations were conducted to determine sample size and treatment time reductions for multiple enrichment strategies. REVEAL 2.0 and PHORA 1.0 were the most precise and identified four statistically significantly different ranked groups for clinical worsening (p <2×10⁻¹⁶), specifically identifying an additional very low-risk group and a high-risk group, which had a much higher incidence rate than the others. The PHORA risk algorithm substantially outperformed NYHA Functional Class. REVEAL 2.0 & PHORA 1.0's risk grouping provided the greatest time & sample size savings for all enrichment strategies. This study demonstrated the value proposition of risk algorithms, including PHORA 1.0 for PAH trial enrichment.

The PHORA model may capitalize on newly completed clinical trial and observational study datasets for extraction of demographic, laboratory, EKG, hemodynamic and comorbid conditions. It is contemplated that modern statistical learning methods for selecting features in high dimensionality of the data, including multiple modalities, may lead to a better predictive model of a clinical outcome without overfitting.

Imaging data: In the present study, the NEDA database served as the main training set for ECHO integration and are presented in Table 2.

TABLE 2
Data sources and expected right ventricular variables

| | Number of | |
|---|---|---|
| Data Source | patients | Expected Right Ventricular (RV) Variables |
| PAH database | 274 | RV End Diastolic Area |
| | | RV End Systolic Area |
| Australian/New Zealand | 5,000 w/≤PAP > | RV Fractional Area Change |
| NEDA database | 50 & No ECHO | Tricuspid Annular Plane Systolic Excursion (TAPSE) |
| (ACTRN12617001387314) | LHD | Right Atrial Area |
| | | RV global, basal strain |
| | | Systolic Pulmonary Artery Pressure (sPAP) |
| | | TPASE/sPAP |
| | | RV global strain/sPAP |
| | | Pericardial Effusion |
| | | RV Velocity Time Interval |
| | | PA Acceleration Time |
| | | TR mild-moderate-severe |
| UK MRI Database (ASPIRE) | 418-526 | RV End Diastolic Area |
| US MRI Registry | 900 | RV End Systolic Area |
| | | RV Ejection Fraction |
| | | Right Atrial Area |
| | | RV EDVI |
| | | RV ESVI |
| | | RV Stroke Volume/Index |
| | | RV mass/Index |
| | | Pericardial effusion |
| | | PA Relative Area Change |
| | | TR mild-moderate-severe |
| Clinical Trials: | | |
| REPAIR (NCT02310672) | 89 | |
| (Actelion/Janssen) | | |
| CS1-003 (CERENO) | 30 | |
| ARTISAN (United | 50 | |
| Therapeutics) | | |
| COMPASS-3 (Janssen) | 100 | |

The PAH Biobank contributed longitudinal data and resources to retrospectively collect 2 ECHO studies (baseline, 4-6 months post enrollment) on 274 diagnosed patients. The US MRI & ASPIRE registries served as the main MRI training set. The REPAIR, REPLACE, COMPASS 3, ARTISIAN & CERENO trials from Janssen, United Therapeutics and CERENO functioned as the validation cohorts.

Adult Clinical Data & Protein biomarkers: Sources of adult clinical and biomarker data may come from the PAH Biobank (n=2017) and from two Bayer trials (REPLACE, n=225: RESPITE, n=61:), a Gossamer trial and from contemporary trials from Liquidia (INSPIRE, n=153:), Gossamer (PAH, n=250:), United Therapeutics (Freedom Trials, n=1703: BREEZE, n=45:ADVANCE OUTCOMES, n=700:).

Both biomarker and genomic markers of risk were evaluated, in particular biomarker (ST2, NT-proBNP, endostatin, HDGF, Gal3, IL6) measurements using a custom printed multiplex electrochemiluminescence based ELISA and clinical data obtained from 2,017 adults and 182 children with Group I PAH from the PH Biobank. In some examples, higher ST2 and NT-proBNP levels were associated with increased risk of death (hazard ratios 2.79, 95% CI 2.21-3.53, p<0.001 and 1.84, 95% CI 1.62-2.10, p<0.001 respectively) [31]. In multivariable modeling, serum IL-6 (32) was associated with survival in the overall cohort (hazard ratio 1.22, 95% CI 1.08-1.38; p<0.01). ST2 significantly improved the model (HR 2.05) over REVEAL 2.0 score (HR 1.88). In adjusted analysis using pediatric PAH Biobank samples, addition of ST2 significantly improved the model (HR 2.05). Artificial Intelligence unbiased cluster analysis was used to examine a plasma biomarker alone for survival risk using the PAH Biobank samples and clinical data (FIG. 9). As shown biomarkers alone produced four clusters and these clusters reflected a spectrum of mild to severe survival risk. It is contemplated that addition of additional blood biomarkers to the PHORA model could improve severity/survival prediction.

Genomic biomarkers: The adult genomic data was derived from US Pulmonary Hypertension Scientific Registry (USPHSR) and PAH Biobank (whole exome sequencing; n=1886), and an additional data source with GWAS (n=911) and whole genome sequencing (WGS) (n=325). Through these sources, the study identified common and rare single nucleotide variants, CNVs, structural variants, non-coding variants detectable in WGS and GWAS data, where these features combined filled in some of the missing genomic influences that is predicted to be present in PAH. Imputation was performed where necessary [39]. Variants associated with patient survival were identified [40], and associations with aggregate variants via pathways were identified. It is contemplated that careful feature selection and feature combinations guided by PHORA algorithm to differentiate patient survival improves the ability to find meaningful results.

Example #4—A third implementation of the PHORA model (PHORA 3.0): In the following example, PHORA 3.0 was implemented for adult demographic groups using an ensemble strategy. The PHORA model ensemble included multiple modules: clinical, genomic, biomarker, imaging (ECHO/MRI), and potentially others (e.g., EHR). Each module was built separately, but all followed the steps of feature selection, model building (i.e., training using a TAN), and prediction of three clinical outcomes (survival, clinical worsening, and PAH-associated hospitalization).

More specifically, for each module, the corresponding complete data was used for building the structure, whereas the model parameters may be learned using all data, with missing data processed using an Expectation-Maximization algorithm [41]. The modulization and ensemble approach is extremely flexible, allowing for other data (e.g., EHR model) to be integrated. Further, missing data or different types of data available at different locations are handled more efficiently.

After each module was built, the study determined ensemble models for prediction. Depending on which types are available, the study determined the weights of the relevant modules in the ensemble through cross-validation to minimize a cost function. Prediction accuracy of outcomes is a natural measure of cost; it is contemplated that other performance measurements may also have been considered, including the Brier score or even more complex cost functions weighing the two types of errors (false positive/false discovery (1-precision) or false negative (1-recall) of 1-year survival) may also have been constructed with input from physicians.

For improved structure learning and expansion of the complete dataset, the study used appropriate imputation methods for missing data (e.g., Michigan Imputation Server for imputing missing genotypes. As all imputation methods have weaknesses, as a data quality control, patients missing more than 50% of data for a given model (clinical, genomic, image model) were not used for structure learning of that specific model, but the EM algorithm seamlessly allowed for these patients to be used in parameter learning. The success of the imputation method was determined by the cross-validated accuracy of the structure after parameter learning, and structures were “averaged” using multiple imputation methods to improve generalizability. A major hallmark of Bayesian networks is their ability to make intelligent predictions even with missing data, however imputation was not done during final model testing.

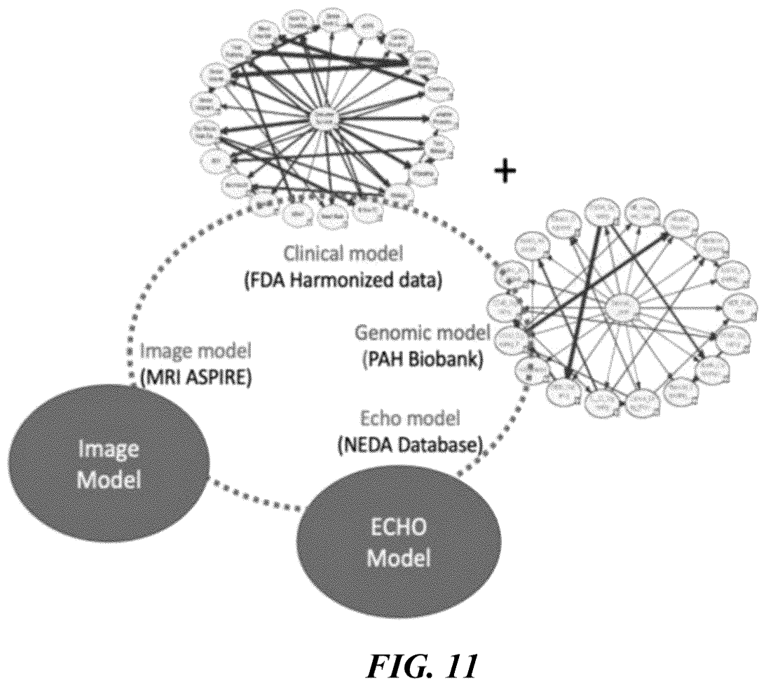

An example prototype ensemble model is shown in FIG. 11. The prototype ensemble model utilized information from two modules, clinical and genetics if information from the other modules are not available for prediction. In the following three subsections, using the clinical module as an example, two specific steps are described for building the clinical model: (1) Preliminary features selected from each pharmaceutical company will be combined and subjected to a rigorous machine learning method for final feature selection using the FDA harmonized dataset; (2) A tree augmented naïve Bayes (TAN) model will be trained based on the selected features to predict patient outcome. Finally, to ensure the generalizability of the trained model, the study may use the most recent clinical and registry data (post-2015) from a broad source to achieve appropriate representation.

Feature selection: The study collected a list of the preliminary risk factors in PAH from experts and literature ranging from sex/gender, NYHA FC, demographics, hemodynamics, labs, biomarkers, imaging to comorbidities. The list was sent to each pharmaceutical company for initial analysis on the clinical trials, prior to or concurrently with clinical trial subject-level data harmonization at the FDA. It is contemplated that initial feature screening may be alternatively conducted in each data set using the significance of the univariate Cox proportional hazards. Then the list of variables for further feature selection was summarized.

Feature candidates from different sources were subjected to a rigorous feature selection process using a suitable machine learning procedure based on the harmonized FDA data [42-44]. Given the potential of multicollinearity (i.e., confounding of variables) and the high dimensionality of the data, suitable machine learning methods with simultaneous feature selection were preferred, as they led to good predictive power without overfitting. For datasets with unique enriched data do not present in the clinical trials (imaging, biomarkers, etc.), features based on publications and expert opinion were chosen.

Model building and prediction: Bayesian network models were built with or without discretizing features with continuous measurements using software packages such as GeNIe [45] and bnlearn [46]. In particular, the structures of TAN models were learned, estimating their parameters for predicting the probability of patient death at one year, as described above. Again, for datasets with unique enriched data do not present in the clinical trials, the study trained separate TAN models in the largest available datasets (e.g., NEDA for ECHO, ASPIRE for MRI, PAH Biobank for biomarkers, etc.). A primary model learned in harmonized FDA clinical data was created with additional secondary models that account for unique type features (Imaging, genomics, biomarkers, etc.). The primary and secondary models were combined using a multimodal ensemble strategy [47, 48].

Evaluation plan: Cross-validation was performed while training the individual classifier and the ensemble models. External datasets were used as validation sets to evaluate how well the models performed on completely unseen data.

Software development for PHORA CDSS: The PHORA CDSS provided a unified platform for various PAH risk calculators: REVEAL 2.0, REVEAL Lite2, and one or more embodiments of PHORA models (2.0, 3.0). The predictive algorithm was incorporated into a software function that received the required variables via a form interface and was an engine to calculate risk scores across various models in the CDSS. This function was provisioned via an API (Application Programming Interface). Enhancements using human-centered design methods, such as contextual inquiry, can examine the clinical decision-making processes, identify contextual barriers, and improve the design to solve any barriers. In an independent survey, physicians reported the need to better communicate risk, as well as situating the patient's risk in a historical context. Such insights led to design improvements for PHORA CDSS, as shown in FIGS. 2 and 3 contrasted to the older designs shown in FIG. 8.

FIG. 3B includes features that are configured to provide feedback to physicians about the importance/influence of particular variables for the risk calculation, which can act as recommendations for physicians to provide more data to have a better prediction for risk. Other enhancements to the app include a longitudinal chart of survival rates over various time ranges like monthly, quarterly, and yearly; the ability to run scenarios/simulations by editing variables values, treatment guidelines, and decision support alerts.

Electronic Health Record (EHR) Integration: In one example, the PHORA CDSS was integrated into clinical workflow by accessing EHR to import the values for the required variables to calculate the risk score. The EHR integration was implemented using contemporary standards like Fast Healthcare Interoperability Resources (FHIR) [41], which offers a web service-based platform for data exchange and interoperability. FHIR implementation offers application programming interfaces (APIs) that can map to patient-centric clinical entities like demographics, diagnosis, labs, and procedures. In comparison to other standards like Health Level 7 (HL7), which has variations among different EHR systems (i.e., Epic, Cerner), FHIR provides a common integration platform.

It is contemplated that linkage to the maximum number of variables from EHR may help in increasing the efficiency of the use of the PHORA CDSS in the clinical workflow.

In the third implementation of PHORA (PHORA 3.0), discretized feature variables that could not be harmonized were retained as continuous variables whenever possible [51-53].

Deep learning methods, including neural networks and convolutional neural networks with multiple hidden layers, were used to build the PHORA model, with care taken to guard against overfitting. It is contemplated that greater than 1-year survival prediction accuracy with the PHORA 3.0 model using only clinical data can be achieved using multiple metrics to measure accuracy of survival prediction, including AUC, Brier scores, and precision recall. The study had successfully identified genomic variants and pathways for building the genomic module for the ensemble model. The study retrained the genomic module with a large sample size, starting with variable selection and pathway identification and different discretization strategies or treating the features as continuous variables. The ensemble approach with the cross-validation weighing scheme may upweight or downweight models from each module accordingly depending on their informativeness for survival outcome prediction. The alternative strategy may also be applied to other modules in our ensemble model, including imaging data.

Genomics Work: A meta-analysis of GWAS results was conducted between the PH Biobank (n=1,885) and another dataset (1R01HL134673) (n=911) [33]. After adjustment for age, sex, prostacyclin use, and PVR, self-reported Hispanic patients were observed to exhibit significantly improved survival versus NHWs (p=0.009) [34]. The study extended these evaluations to determine independent genetic determinants of survival. GWAS data from AHN and PH Biobank (PAHB) were processed and cleaned at Indiana University using GWASTools-based pipeline. Logistic regression was used with survival outcome as a dichotomized outcome variable. GWAS of the outcome was conducted separately in AHN and PAHB, then a meta-analysis of the two cohorts was conducted. The study identified one survival loci (NCKAPL1, p-value<5×10-8) that represents potential target for validation. Once validated, this locus was used to stratify risk PAH patients with PHORA 3.0. Whole-genome sequencing was performed on stored samples from 221 PAH patients. Samples were included with Long survival greater than 7 years and Short survival (<5 years). Variants were filtered for quality, assigned to genes, and filtered for function and population frequency. Genes are grouped based on Canonical Pathways defined in Ingenuity Pathway Analysis.

Of the pathways containing more than one gene mutated in 3 or more samples, 29 were observed to be associated with survival length. Biologically relevant pathways include Pentose Phosphate (p=0.005), IL-22 (p=0.006), Phospholipase C signaling (p=0.007), Endocannabinoid related pathways (p=0.01), and Thioredoxin pathway (p=0.015). A Neural network model based on the top pathways was constructed (FIG. 10) that predicted Long/Short survival.

EKG work: A meta-analysis was conducted on the results of univariate Cox's analysis (mortality at baseline with ten EKG variables) from SERAPHIN, BREATHE-1, and PATENT-2. The study showed that non-sinus rhythm (p=0.018) and mean ventricular rate (p=0.001) were most predictive for higher mortality. Presence of atrial or ventricular extrasystole were predictive of higher survival (p=0.004).

Example #5—PHORA 3.0 with pediatric patient datasets: A contemporary risk prediction model was disclosed for pediatric PAH patients (PHORA PEDs). Previously, none of the adult-based PH risk prediction scores were customized or validated for pediatric patients [16]. For example, many of the REVEAL clinical variables were either not collected in young pediatric patients (e.g., 6MWD, pulmonary function testing, etc.), or include inappropriate disease types (APAH-CTD) or age cut-offs (>60 years of age).

Pediatric PH is also complicated by developmental causes or congenital malformations and compounded by growth. The pediatric PHORA can be created from a harmonized, subject-level dataset of pediatric clinical trials from FDA and the Pediatric Pulmonary Hypertension Network registry, which includes 13 of the top pediatric PH centers in North America. Over 1,500 pediatric PH subjects were enrolled into the PPHNet Registry, which includes detailed longitudinal clinical phenotyping. PPHNet registry data is housed by the Data Coordinating Center at Boston Children's Hospital. PPHNet supports ongoing studies with members of the PPHNet for diverse studies of pediatric PH.

PHORA PEDs development parallels the development of PHORA 3.0 for adult PAH patients and can be built similarly using machine learning methods for variable selection, predictive modeling, and data integration, taking into account of the potential confounders for pediatric patients described above. The PHORA CDSS can be configured to present the specific needs of pediatric clinicians.

Pediatric data collection. Sources of pediatric biomarker and clinical data can come from the PAH Biobank (n=182, 16 [9%] with death or transplant), the PPHNet Registry (n=1475, 149 [10.1%] with death or transplant) and United Therapeutics (Trials, n=337); Actelion (Trials, n=1,304; Observation studies with pediatric enrollment: ); Bayer (Trials, N=24). Clinical trials noted above can be used when harmonized with preexisting clinical trial data at the FDA.

Pediatric PHORA model (PHORA PEDs): In another implementation of the PHORA model, a pediatric PAH risk model can be configured and trained using the PPHNet data, following the same steps of feature selection, engineering and refinement, and modeling building and validation.

Feature selection: Feature selection was guided by pediatric clinical experts (PPHNet) and conducted through individual pediatric clinical trial datasets. The candidate features can be combined, and a further rigorous feature selection process was conducted using machine learning algorithms with the pediatric clinical trial data harmonization at the FDA. This step mirrors that in building the adult PHORA model to minimize potential confounding and overfitting. Using the same method used for the adult feature selection and model refinement but using the PPHNet data. A selection of pediatric variables can be identified as shown in Table 3.

TABLE 3
Univariate risk factors for death or
transplant include (PPHNet Registry)

Older age at PH diagnosis
Pediatric Functional class III/IV
Higher HR-for-age-z-score at enrollment at diagnosis
Higher mPAP, diastolic PAP, RV systolic pressure and pCO2 at
diagnosis
On diagnosis ECHO, greater degree of septal flattening, higher
peak RVRA gradient, peak TR regurg velocity, LVSD and RVDD,
ratio of RV:LV diastolic dimension
Higher NT Pro-BNP
Higher number of PH medications at diagnosis

Several features, such as not having congenital diaphragmatic hernia (CDH) and growth measures, are pediatric-specific. Where features are predictive for both adults & pediatric patients, feature engineering from the adult dataset may be directly leveraged by translating cut-points to “z-scores” (number of standard deviations from the mean value) that each cut-point represents. The z-score in children can be calculated using a different mean/nominal value and standard deviation that is appropriate for pediatrics. This allows an increased sample size for feature engineering [54]. Features that are continuous variables can be used directly without discretization.

Model building and prediction: Once features were selected (and cut-points were determined if preferred), a TAN model can be built based on the primary training dataset (PPHNet). Harmonized pediatric clinical trial data can be reserved as a validation dataset, updating parameters if needed. Finally, testing can be conducted in the pediatric observational study datasets (OPUS, OrPHEUS, Bayer, JPMS-PAH, EXPERT & PAHBiobank). Datasets were organized as such to maximize sample sizes for training and validation sets, reserving smaller sets for testing.

Evaluation Plan: Cross-validation can be the primary tool for evaluating the model-building component, with one-fold of the data as a hold-out test set and cycling through successively. This strategy may ensure maximal usage of the data without incurring overfitting. The final model built can be further validated with independent datasets that have not participated in the model building.

Example #6—Incorporation into clinical workflow. In some embodiments, the study identified practical implementation of regular risk assessment, including provider time constraints to enter multiple variables into a risk score calculator. Accordingly, the clinical data-points for PHORA 2.0, 3.0 and PHORA PEDs can be imported directly from the EHR. The data can be updated dynamically as new diagnostic information becomes available or changes and will issue an alert if relevant changes occur in key variables or outcome probabilities. This streamlines the integration of clinical workflow, both during a patient-physician appointment as well as through remote, collaborative decision-making. Features and visual enhancements were built into PHORA CDSS that facilitate improved uptake, communication, and usability by health care providers. These Features and visual enhancements were based upon a series of human-centered design methods, such as contextual inquiry with domain experts. One such feature included a “What If” capability that enables physicians to modify or add any clinical variable of the PHORA model in the CDSS web application to run different scenarios. The user can customize both the layout of the interface and the structure of underlying decision logic to accommodate professional preferences, per FIGS. 3, 8 & 13)

Applying PHORA 1.0 to clinical enrichment strategies: The FDA advocates the use of prognostic enrichment of clinical trials by preselecting a patient population with an increased likelihood of experiencing the trial's primary endpoint. The study compared validated clinical scales of risk (COMPERA, French score, REVEAL 2.0 and PHORA 1.0) to identify patients that are likely to experience a clinical worsening event for a trial [25, 26]. Power simulations were conducted to determine sample size and treatment time reductions for multiple enrichment strategies. REVEAL 2.0 and PHORA 1.0 were the most precise and identified four statistically significantly different ranked groups for clinical worsening (p<2×10-16), specifically identifying an additional very low-risk group and a high-risk group, which had a much higher incidence rate than the others. Risk algorithms substantially outperformed NYHA Functional Class. REVEAL 2.0 & PHORA 1.0's risk grouping provided the greatest time & sample size savings for all enrichment strategies. This study demonstrates the value proposition of risk algorithms, including PHORA 1.0 for PAH trial enrichment.

Applying PHORA 1.0: PHORA 1.0 may be applied to define the benefits of dual combination therapy in low-risk patients [27]: Application of risk stratification to the AMBITION clinical trial data has been previously published [28]. The study hypothesized that more discriminatory risk models like PHORA 1.0 might be able to discern a group of low-risk patients that did not benefit from upfront dual combination therapy. In collaboration with the FDA, the study applied both risk algorithms within the AMBITION clinical trial to identify if upfront combination therapy truly provided a significant benefit within all risk groups [27]. ROCs were generated for REVEAL 1.0, REVEAL 2.0 and PHORA at baseline and 16-week reassessment to determine their ability to predict one-year survival from the time of assessment.

Treatment effect was re-analyzed per risk group using the trial's original primary endpoint, as well as time to all-cause death censored at one-year. PHORA was observed to be more discriminatory than REVEAL 2.0 at the 16-week reassessment for predicting clinical worsening at 1 year, thus providing the first validation of PHORA 1.0 in a contemporary global dataset. The low-risk groups of REVEAL 2.0 (≤6) and PHORA (<5%) did not have significant benefits in time to clinical failure.

Both of REVEAL 2.0's (≥9) and PHORA's (>10% risk) high-risk group; however, did see significant treatment benefits at 1-year (HR=0.49, p=0.008, and HR=0.44, p=0.007). Within PHORA's low risk group, a greater number of special interest adverse events were experienced in the combination therapy group versus monotherapy. Thus, risk stratification, using PHORA 1.0 can identify low-risk groups that would not achieve significant benefits on combination therapy versus monotherapy. This is one example of how PHORA will assist clinical decision-making regarding upfront treatment benefit versus cost/potential for adverse effects.

As shown in Table 2, hemodynamic, non-invasive and laboratory measures were identified in univariate analysis to be significantly associated with survival in pediatric PH. Together with the cut-offs for the identified variable, a TAN-based PHORA model was developed using the pediatric datasets and feature selections. In the example PHORA model for pediatrics, continuous variables were used when possible, to build the model.

Neural network and convolutional neural network modeling strategy with potentially multiple hidden layers were used to produce the predictive model. Good practices to guard against overfitting were exercised. When additional modalities of data, such as imaging, were available, they were integrated with the clinical model to strengthen the predicted power of PHORA-PEDS.

Validation of the PHORA-PEDS model was tested initially using the 187 children enrolled in the PAH Biobank, and the model was refined as necessary for optimal survival outcome prediction. Validation can be accomplished by longitudinal enrollment from PPHNet clinical sites. Typical enrollment in the PPHNet registry was approximately 200 participants a year. This can provide the adequate participants to formally validate and refine PHORA-PEDS for optimal performance.

The study may advance the PHORA CDSS web application can be configured to support use by physicians under multiple sites 1020. The application may be securely hosted at private system and provide secure authentication and authorization schemes to segregate access and data visibility by each site. Essentially groups of physicians affiliated with a site can only view their own site's patient records. The database may be a relational database allowing the identification and linkage of patient data across multiple sites. A consolidated MR registry may be created by aggregating data across all sites leveraging the data sources as PHORA CDSS and supplemental Data Entry Portal.

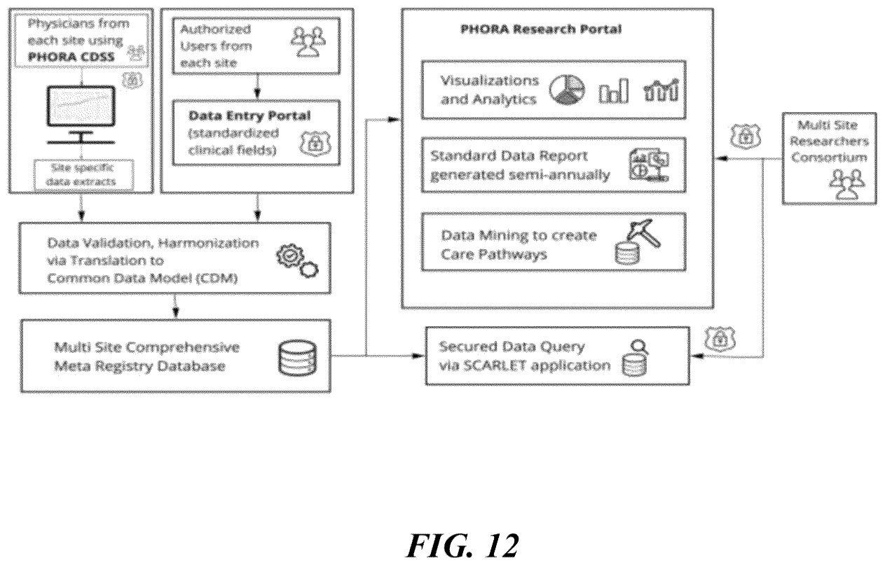

Registry Data Model: The PHORA CDSS application (FIG. 12) comprising the essential variables for the risk calculation provides the study with the baseline database, which covers patient attributes like demographics, vitals (HR, BP), and labs like NT-proBNP. The variables under PHORA CDSS may be one of the data sources to the registry.

The database can be augmented by additional data elements to cover interventions and outcomes. The supplemental dataset may include medications, palliative care, surgical evaluations, and procedures like transplants, hospitalization related to conditions like Syncope, Dysrthymia, etc. Each investigator participating in the registry may choose whether to use predictive algorithms (REVEAL & PHORA 1.0/2.0 scores) or not for their treatment decisions. Demographic, functional, diagnostic, laboratories and outcomes may be recorded at entry into the Meta registry at regular intervals.

The meta registry can be supplemented by data mining and visualization techniques. It is contemplated that the diverse set of data elements may lead to data harmonization and the creation of a standardized common data model that can be used for both data persistence and consolidation into a central meta registry. A relational database schema may be developed that stems from PHORA CDSS and the collection of supplemental clinical data elements. This schema will evolve as the PHORA Common Data Model (PHORA-CDM). Each site's data may be persisted under the standard schema of CDM to allow consistency in its use for analysis.

Data Entry Portal: The PHORA CDSS may include a data entry portal using human-centered design methods to collect data across the different clinical sites. The portal may employ authentication and authorization methods for secure ingestion of supplemental data from each site.

Designated authorized users from each site may be able to enter data into electronic forms. This portal may implement validation on fields at the entry-level to avoid errors as much as possible. Another advantage is the direct linkage to registry's CDM-based database which avoids extra processing before storing the data. When the data is received from various sites, the study may develop a data cleansing and harmonization process before loading it into the final comprehensive PHORA registry database. The study may also implement a data deidentification process that concurrently saves the data in the de-identified format at the time of entry.

The standard methodology can quickly scale during the adoption of PHORA at multiple clinical sites via creation of secure accounts linked to a site and without requiring local deployment at each site. The centralized deployment of PHORA CDSS supporting multiple sites and data entry portal based on their authentication and authorization scheme reduces the burden of instantiation of infrastructure at the site level and increase the plausibility of sites participating in PHORA research network.

Data Harmonization and Aggregation: The standardization of data elements can be accomplished using a CDM across the two sources: PHORA CDSS and data entry portal. The meta registry may be built by consolidating all the site-specific data into a central database via an ETL (Extract Transform Load) process. This process may also harmonize any variations encountered at the time of data entry so that the MR registry data field values are standardized as much as possible. Thus, metrics can be retrieved for visualization, analytics, and reporting. ETL may be automated to make the process robust and scalable.

PHORA Research Portal: a secure baseline infrastructure was developed to support large-scale communities of practice style functionality. The portal includes features like document sharing and community announcements, all supported by a custom-developed identity authentication and access management system to operate in a multi-site consortium environment. It is contemplated that a PHORA Research Portal will provide a secure enclave to disseminate the research outcomes like dashboards, reports, events, and updates. The portal may include a content management system, accessible only to consortium members and used to organize and maintain consortium documentation. Content will be organized into sections by workgroup, and authorized users will manage uploads.

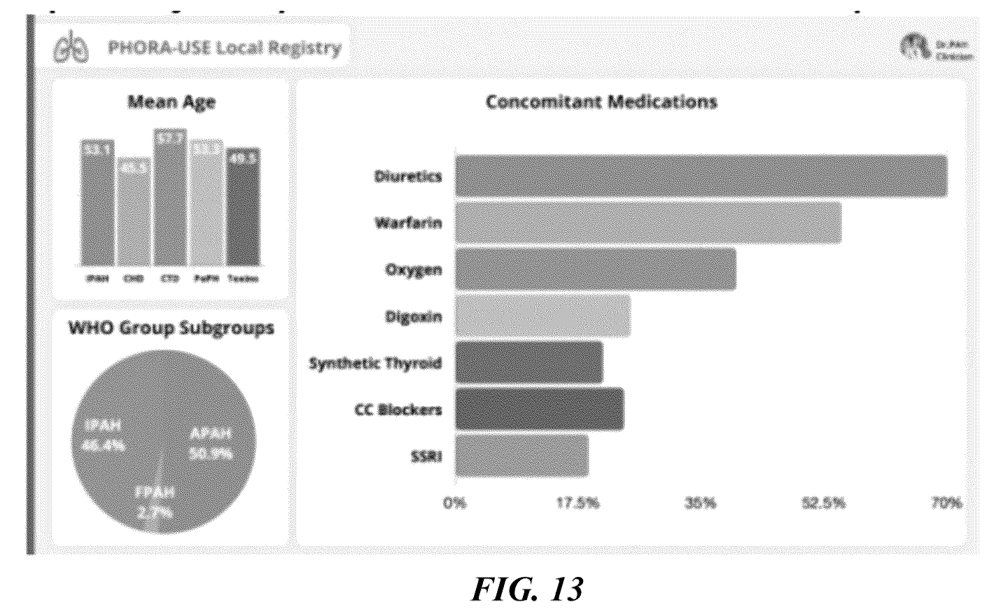

Visualization and Analytics: The data from the MR registry may be used to create dashboards around clinical metrics that will be site specific as well as collated across sites. By analyzing data collected, the user-centered design methods can be utilized to design a Local Registry visualization tool that will allow registry users to get meaningful statistics about their clinical site's population (FIG. 13), such as demographics, comorbidities, PH drug use, and risk status.

Cohort Analytics via Visual Analytics may be leveraged to allow clinicians to uncover correlations between patients' risk/attributes [52]. The consolidated registry may also have additional benchmark parameters to show comparisons across different participating sites (e.g., mortality, clinical worsening, hospitalization & achievement of low-risk status). The various visualizations may be embedded in the PHORA Research Portal and centrally accessible by the multi-site consortium in a secure manner.

Reporting: The aggregated meta registry may implement processes to generate reports of the metric at a frequency semi-annually. The report will list the metrics' value at a local site and its comparison over an aggregation at all other participating sites (FIGS. 1 and 11) and as elaborated upon above. These reports will be available via the research portal.

Hosting, Access Control and Data Security: The software components at the data center may be hosted on the server and database inside a secure firewall. All the application servers and databases may be kept physically separate to enhance security. All communication may be secured by transport layer security under the SSL (Secure Socket Layer) protocol. An Identity, and Access Management Service may control user management and data accessibility in order to allow only authorized users to view and enter data, using a centralized authentication provider using industry standards like OAuth 2 [55]. Each site could have its separate staging database to store de-identified data before aggregation to the meta registry.

Data Query and Extracts: A disease-specific registry platform may be employed (known as SCARLET, Scalable Analytics Registry for Rapid Learning and Translational Science). The platform may include a query interface component that can allow for secure access to a registry database similar to PHORA. SCARLET can be linked to any schema across multiple database types, allowing easy accessibility at various stages of data persistence. The query engine may include an intuitive user interface where queries are generated and can be saved to a library where they can pre-run and cached following the data refresh to allow for quick access to the new results, as well as shared with other researchers within a project. The de-identified data extracts can be disseminated via the PHORA research portal acting as the hub for dissemination of all research artifacts. The registry may be used to create an opportunity for data mining along with the development of machine learning to employ patient-centric treatment regimens and improve clinical outcomes. The study can leverage tools like SCARLET to create data sets fed to PHORA Pathways modeling.

Example #7—Risk Profiling Registry: The study may create a multi-center National adult and pediatric “Risk Profiling” Meta registry for PAH clinicians. A serendipitous consequence of the efforts to harmonize data from multiple registries may be to generate a multi-center, National Meta registry for adult and pediatric PAH. Essentially, each PHORA CDSS may serve as an individual site's local PAH database (PHORA-USE registry), equipped with simple tracking and analysis capabilities to a provider site-specific quality initiative projects and research. Deidentified data from each participating site may be periodically extracted & loaded to the Meta registry housed at the data center.

The working registry can objectively track risk scoring performance using the PHORA CDSS, correlated with interventions and outcomes for participating centers. A participating site may receive a Data Quality Report and Quality Assurance Report from the PHORA-USE registry that may provide each site with a summary of key data they have entered into PHORA-USE and highlight any inconsistent and improbable data values. This may also allow the sites to analyze their risk-based treatment patterns that can be benchmarked against others and act as a useful tool for auditing.

These comparative metrics can range from patterns of drug usage (titration rates, drug combinations, etc.) and timing of transplant referrals employed in response to various levels of risk to pure outcome measures (attaining low risk status, hospitalizations, etc.). It is envisioned that the comparative reports may facilitate the maturation and refinement of site-specific risk-stratification behaviors of low performers & elevate outcomes with other sites. Ultimately, the PHORA-USE registry may have the potential to become the de-facto, PAH registry which can be queried for research, a tool for benchmarking and to allow machine-learned modeling to create best-practice patterns and guidelines.

PHORA Pathways Modeling: PHORA-USE Meta Registry may be used to provide a data-driven analysis of PAH across institutions and nationally. It is contemplated that the observed events reported to a PHORA-USE Registry may provide a unique opportunity to analyze PAH progression pathways, to better understand treatment patterns of risk stratified patients, understand how PAH evolves over time and validate the benefits of risk-based treatment outcomes.

Mining Data from PHORA-USE Meta Registry: Innovative data mining and visualization techniques may be used to eludicate emerging patterns in the registry data. The data mining techniques may handle the real-world properties of registry data, such as handling event concurrency, multiple levels-of-detail, temporal context and patient outcomes [17]. Such techniques have been previously evaluated on a variety of conditions, such as patients with lung disease developing sepsis and hyperlipidemic patients with hypertension and diabetes pre-conditions [17].

Prior to the PHORA Meta Registry data collection, the mining algorithms may be validated by extracting meaningful patterns from the REVEAL Registry (N=3515) and the PPHNet Registry (N=1000), which will represent “prerisk-based treatment outcomes”. This may ensure the data analysis pipeline is ready when the PHORA Meta Registry becomes online and allow meaningful comparisons with ‘Risk-based” treatment outcomes in the Meta registry.

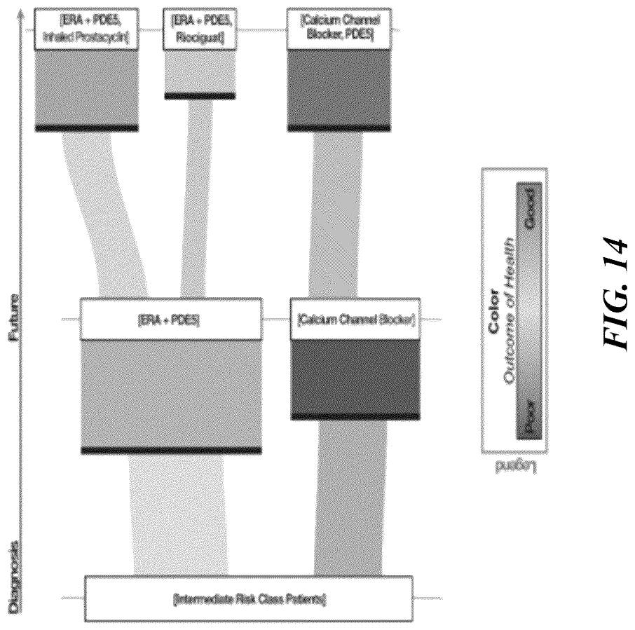

Visualizing and Evaluation of PHORA Pathways: After meaningful patterns are extracted from the PHORA-USE Registry, the common frequent event patterns may be visualized to provide overviews of the registry. For example, the CareFlow visualization technique [57] shows PAH treatments as nodes positioned alongside the horizontal axis, which represents the sequence of treatments (FIG. 14).

In FIG. 14, green nodes represent positive outcomes, whereas red nodes represent poor outcomes. Node height represents how many patients received the treatment in that particular sequence. Node width represents the duration of how long it took the patient to transition to this treatment. In FIG. 12, patients took calcium-ion Channel Blockers more quickly than ERA+PDE5.

Sequences of treatments are linked by edges, i.e., about ⅔ of patients that took ERA+PDE5 followed this up with Inhaled Prostacyclin. These patients had a better outcome (greener) than those that took ERA+PDE5 alone. While this shows an analysis of treatment outcomes, other possible correlations can also be evaluated. The pathways correlated with positive & negative outcomes can be then validated in a separate cohort of registry users & shared with the PH community for future guideline development.

Expected results, caveats and alternatives. Clinical validation of PHORA 3.0 and risk prediction-based outcomes can be achieved in this registry as demonstrated by repeated application of risk prediction strategies as part of the process of clinical care, which leads to improved results. This validation strategy requires investigators applying risk prediction models prospectively in a new population as “a rule” as opposed to a statistical validation. By allowing some investigators to choose a non-risk-based approach, direct comparison of outcomes can be evaluated.

Example #8—Treatment Roadmap: Machine-learned, best practice treatment roadmaps can be created, using innovative data mining and visualization techniques to inform guidelines for effective PAH management. The exploration of temporal knowledge from longitudinal EMRs with data mining techniques is an important problem that has been the focus of study for much medical informatics research. In this application the study may capitalize upon innovative analytics to mine frequent patterns and displays them in the visualization alongside meaningful statistics. In addition to visualizing treatment-related outcomes, it may allow the profiling of differences in PH management among regions. In turn, this tool may identify outcomes that are linked to differences in risk profiling behavior, regional levels of awareness of PAH treatment options, health care provider systems, environmental and geographic factors and use of/availability of specific PH medications. Leveraging the magnitude of data in PHORA-USE Registry will permit investigators & hospital administrators to ask questions beyond the scale of local registries, including “built-in” cohorts for cross-validation. Machine learned treatment patterns resulting in best outcomes (i.e., dark green pathways shown in FIG. 14) may then be validated in a separate cohort and shared with the PH community for consideration of future guideline development. PHORA-USE will also provide a data base for planning clinical trials.

Experimental Results and Examples: Enhancement of PHORA with contemporary adult and pediatric clinical trials and registry data inclusive of enhanced imaging, biomarker and genomic data will result in superior risk stratification and discrimination of outcomes.

The sensitivity and specificity of PHORA predictive algorithms will increase with additional data mining of contemporary clinical trials and registries that include modern biomarker, genomic and imaging parameters.

A PHORA predictive algorithm for pediatrics can inform clinician treatment decisions in a manner similar to adult PAH.

Tracking risk stratification usage (REVEAL, PHORA, PHORA PEDS) and performance amongst providers can inform patterns of treatment decisions and improve provider behavior and patient outcome. Understanding the drivers of provider risk profiling behavior will facilitate behavioral change through feedback intervention and machine learned “best practices” enhancing global uptake of risk stratified treatment interventions and providing an alternative pathway to validate these interventions, outside a costly randomized clinical trial.

The disclosed PHORA CDSS improves available resources for physicians to identify individualized treatment sequences that minimize patient risk/optimize outcomes, and improved guidance for care teams to effectively manage costly interventions according to patient-specific risks.

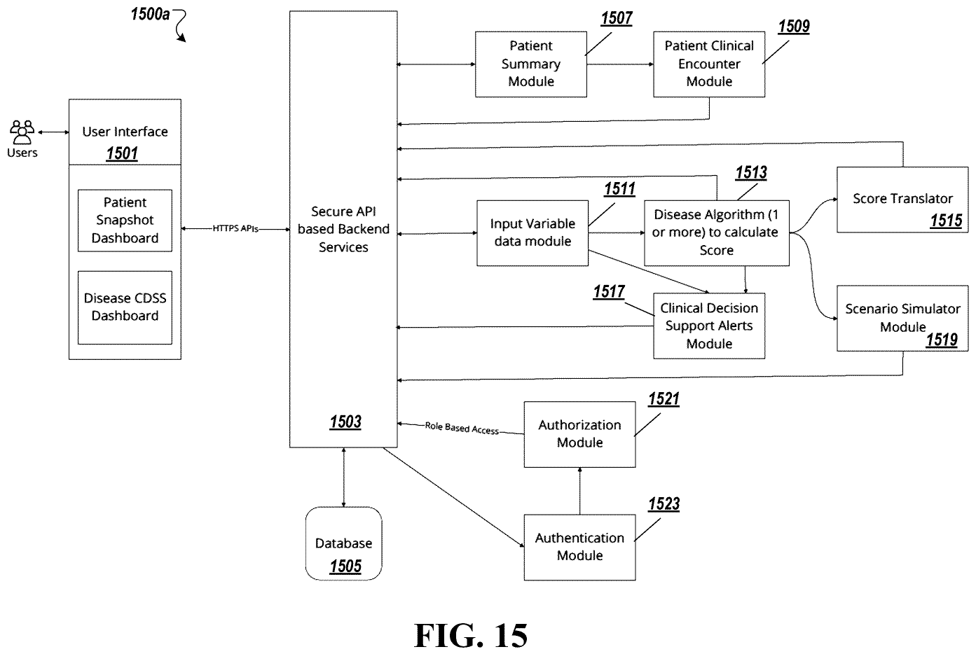

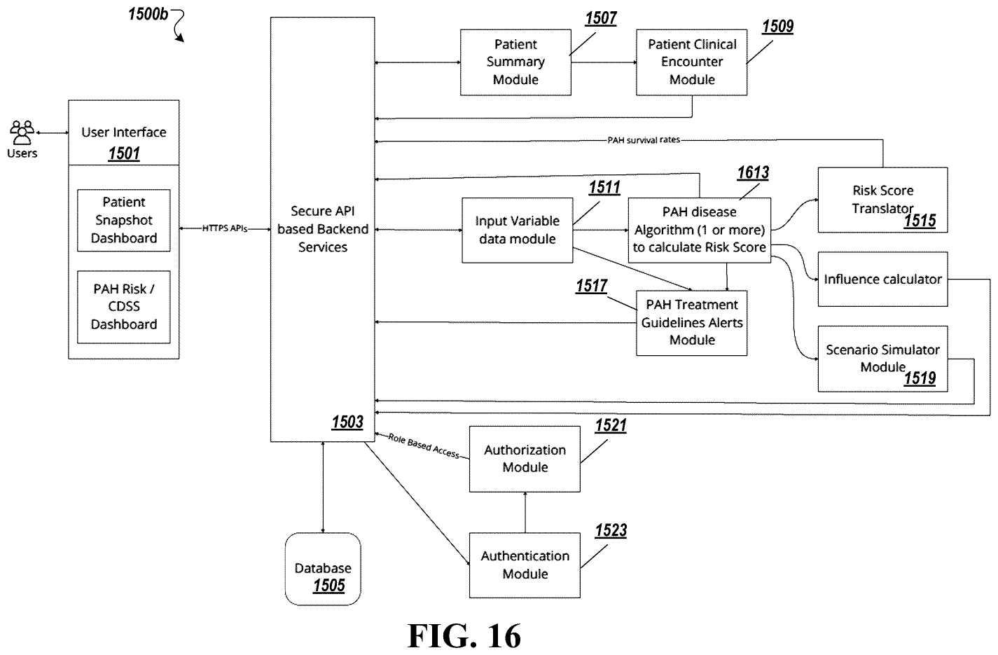

Example #9—Clinical Decision Support Tool. FIGS. 15 and 16 show Clinical Decision Support System 1500 (shown as 1500a and 1500b) (CDSS) to provide a comprehensive system to capture a patient's clinical encounter and observation data to inject into a risk calculation algorithm to align with alerts that can support physicians to make clinical decisions for treatment regimes. A software architecture and framework are created with functionality-specific modules to create a CDSS system 1500a for a disease area. In particular, the CDSS 1500a may be configured for a particular disease area by incorporating one or more disease algorithms corresponding to a particular disease area. One example of a disease area includes Pulmonary Arterial Hypertension, e.g., shown as 1500b. Other disease areas may be supported.

As shown, the CDSS 1500a includes several modules and components, including, but not limited to, a user interface 1501, an API 1503, a patient summary module 1507, a patient encounter module 1509, an input variable data module 1511, a disease algorithm component 1513, a score translator 1515, a clinical decision support alters module 1517, a scenario simulator module 1519, an authorization module 1521, an authentication module 1523, and a database 1505. More or fewer modules and/or components may be supported. All of the modules may communicate with each other directly or through the API 1503.

Patient Summary Module 1507. The patient summary module 1507 captures the basic demographics of a patient like name, date of birth, medical record numbers, and a primary diagnosis concerning the particular CDSS disease area being investigated. The patient summary module 107 has a linkage to all other data sets relevant to the patient.

Patient Clinical Encounter Module 1509. A patient's visit or encounter with a clinical system or hospital generates data relevant to their disease area or disease areas. The module 1509 reads or captures specific data elements that are key to the processing of data and algorithms for CDSS. Depending on the embodiment, the Patient Clinical Encounter Module 1509 may collect relevant data via a questionnaire that is provided to the patient or supervising medical profession (e.g., patient weight, blood pressure, and any observed symptoms), or may receive data from one or more diagnostic or measurement devices (e.g., electrocardiogram, pulse oximeter, or connected scale). Other data sources may be supported.

Input Variable Module 1511: The input variable module 1511 may be a risk calculator that works as a mathematical function on a list of data variables. The input variable module 1511 captures and normalizes (if required) all the required variables that can feed into the disease algorithm module 1513.

Disease Algorithm/Score Calculator Module 1513. This module houses the algorithms for each disease area that is supported by the CDSS 1500. Each disease algorithm takes as an input a different set of input variables from the input variable module 1511 to emit the score for a particular disease area. Example disease areas include Pulmonary Arterial Hypertension. Other disease areas may be supported. As researchers develop new algorithms to calculate risk for different disease areas, the new algorithms may be uploaded to the Disease Algorithm/Score Calculator Module 1513. In addition, as improvements are made to disease algorithms, the algorithms stored in the Disease Algorithm/Score Calculator Module 1513 may be easily updated by a user or administrator. In some embodiments, the API 1503 can process multiple CDSS algorithms in a single call where each CDSS algorithm corresponds to a different disease state.

Score Translator 1515. The score translator 1515 may take the output of each disease algorithm from the module 1513 (i.e., raw score) and may translate the output into specific indicators for an associated disease or disease area. For example, 0-6 score level could resonate with a patient's high probability of response to a specific treatment. Other score ranges may be used.

Scenario Simulator Module 1511. The score calculated during or after a clinical encounter is typically rooted in using the actual data of input variables for a patient provided by the input variable module 1511. The scenario simulator module 1511 may allow a user or administrator to change certain input variables and see how it changes the score generated for one or more of the disease states. For example, the user or administrator may change input variables such as the number of minutes the patient spends exercising each week or hemoglobin level, the module 1511 may recalculate the scores using the changed input variables, and the scenario simulator module 1511 may display the recalculated scores to the patient or physician. This may also the physician to recommend lifestyle or medications to the patient that best affect their calculated risk scores.

Clinical Decision Support Alerts Module 1517. Using a combination of data sets like patient's health and clinical-based input variables, diagnosis, and risk score; a series of alerts are developed that are displayed to the end users (physicians or clinical experts) who can use them to assist in making a specific clinical decision on treatment and care. The content of alerts can either be codified from standardized published guidelines under a disease area or developed as an outcome of a new research goal.

Authentication Module 1523 & Authorization Module 1521. The access and use of the CDSS system 100 are protected and secure. The first layer is the authentication module 1523 which is currently a username and password-based credential system tied to every user. In the future, this could be extended by adding a 2-factor authentication step to enhance security. With respect to the authorization module 1521, each user may be assigned a role that determines what areas they can access and what actions they can perform on the CDSS system 100. A disease-specific CDSS system 1500 can define its role and actions for the diverse population of end users.

All the data used or generated by the system 100 is stored either on a relational or a non-relational database. In some embodiments, to provide additional security, the backend service is the only connector to the database to keep it under a secure enclave.

User Interface (UI) 1501. The user interface is the layer by which users and physicians interact with the system 1500. Any processing is avoided as much as possible in the UI layer and instead done via the backend engine/suite of services. These services are accessed by UI via secure HTTPS API 1503 endpoints. At the basic level, two views are built into the system.

Patient Snapshot Dashboard. This screen provides a holistic view of patients recorded in the CDSS system 100. It is intended to communicate to end users the most important information that is useful to review the patient's current status. Apart from the patient's name and date of birth, a combination of data elements can be chosen for display, for example, risk level at the last clinical visit, latest diagnosis, latest medication, etc.

CDSS Dashboard. This view provides in-depth information about a patient's current data in the CDSS system 1500. It includes a longitudinal trend chart plotted using the risk scores calculated at different dates in history using selected or relevant disease area algorithms. It also shows a graph to see the historical data point for the input variable used in the risk calculation algorithm. This view has prospects of scalability to add more widgets that can relay the information provided by CDSS service to end users.

FIG. 16 is an illustration of an example implementation of a CDSS 1500b including a Pulmonary arterial hypertension (PAH) risk module. In particular, the CDSS 1500b is substantially similar to the CDSS 1500a of FIG. 15 with the addition of the PAH disease algorithm model 1613 replacing the module 1513 of FIG. 1. As may be appreciated additional implementations of the CDSS 1500 may be similarly created by replacing the module 1513 with different disease state algorithms. The CDSS 200 is referred to herein as the PHORA CDSS 200.

The PHORA CDSS 200 tool may be developed by leveraging the CDSS 1500a described with respect to FIG. 15 for the specific task of scoring patient risk with respect to the disease area of PAH. Below is the description of customization to each module of the CDSS 100 (if needed) and any additional modules developed for the PHORA CDSS 200.

PAH Disease Algorithm/Risk Score Calculator Module 1613. The module 1613 uses one or more risk score algorithms to generate one or more risk scores for PAH. These algorithms may use data from the input variable module 1511 including BNP/NT-proBNP, predicted DLCO, heart rate, NYHA class, and six-minute walk distance. The risk score algorithms used include REVEAL 2.0 and REVEAL Lite 2. All three algorithms intake different sets of input variables specific to PAH to emit the risk scores. The service API 1503 can process all of the risk algorithms in a single call and return the risk score to the client (user interface 101).

Score Translator 113. The score translator 1513 of the PHORA CDSS 1500b uses a translation of risk score to survival or mortality rate. Each risk algorithm used by the module 1613 may use a different translation of the raw score to the corresponding indicator of survival rate.

Influence Calculator 1620. The module 1620 may calculate the influence or impact of each variable (e.g., input variable) on the output of each risk score algorithm or its translator, which is the survival rate in the case of the PHORA CDSS 1600. The module 1620 offers the advantage to disseminate the weight or importance of each variable towards a change to the final risk calculator algorithm. This is also one form of data point toward the clinical decision support model.

PAH Treatment Guidelines/Clinical Decision Support Alerts Module 1517. PHORA CDSS 1600b uses this module 1517 in the form of Treatment Guidelines. Currently, it leverages the standard published guidelines for PAH disease. Since PHORA CDSS 200 is under a research project, newer guidelines can be embedded into the system.

### Discussion

As pointed out in a recent editorial [13] “ . . . risk calculators should remain important adjuncts to comprehensive PAH care. As additional metrics are identified in future trials and/or ongoing registries, new iterations of existing tools, or new instruments altogether, will be needed. Discovery of relevant serum biomarkers, genetic mutations, cardiac imaging tools, and ethnic, geographic, and other factors as well as new treatments and new management guidelines will ensure that structured risk assessment will require the continued evolution of existing tools.” The PAH community has adeptly and repeatedly refined risk assessment tools in the spirit of optimizing patient outcomes. More of the same will be needed as we look to the future.” PHORA is thus in line with the thoughts and feelings of the PH community and provides an important avenue to respond to the community's technical problems. This is important, as the majority of PAH management in the United States has shifted from academic centers to community practitioners, whom lack the experience in managing these complex patients making prognostication tools essential for timing referrals to specialists [14].

Experts also agree that risk stratification is important and should be done routinely [3]; yet it is poorly adopted in the community. In a survey with United Therapeutics, only a third of community physicians reported using risk assessment routinely and only one-third of those used a formal risk assessment tool. Currently, there is no useful feedback mechanism to change these poor adoption behaviors preventing widespread benefit of formalized risk stratification in PAH. Therefore, a software that provides such feedback on a real time basis would allow clinicians to benchmark their outcomes against the national data, allowing them opportunities to modify their practice patterns to improve outcomes by ensuring proper implementation of guideline-based therapy. These feedback mechanisms can be further enhanced by machine learning tools, to identify the ‘best’ behavior yielding lowest risk and hence best outcome for their patients. Thus, the exemplary PHORA system can provide a contemporary and “informed” CDSS for providers (pediatric & adult) that facilitate rapid adoption into clinical practice and which learns “best intervention patterns” can then be incorporated into practice guidelines.

Machine Learning. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model a pattern in the data. In a semi-supervised model, the model learns a function that maps an input (also known as feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as an input layer, an output layer, and optionally one or more hidden layers. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

Example Computing System. The exemplary system and method may be implemented (1) as a sequence of computer-implemented acts or program modules running on a computing system and/or (2) as interconnected machine logic circuits or circuit modules within the computing system (FIG. 1). The implementation is a matter of choice dependent on the performance and other requirements of the computing system. Accordingly, the logical operations described herein are referred to variously as state operations, acts, or modules. These operations, acts, and/or modules can be implemented in software, in firmware, in special purpose digital logic, in hardware, and any combination thereof. It should also be appreciated that more or fewer operations can be performed than shown in the figures and described herein. These operations can also be performed in a different order than those described herein.

The computer system is capable of executing the software components described herein for the exemplary method or systems. In an embodiment, the computing device may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computing device to provide the functionality of a number of servers that are not directly bound to the number of computers in the computing device. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or can be hired on an as-needed basis from a third-party provider. Some cloud computing environments may comprise cloud computing resources owned and operated by the enterprise as well as cloud computing resources hired and/or leased from a third-party provider.

In its most basic configuration, a computing device includes at least one processing unit (102) and system memory (110), as shown in FIG. 1. Depending on the exact configuration and type of computing device, system memory may be volatile (such as random-access memory (RAM)), non-volatile (such as read-only memory (ROM), flash memory, etc.), or some combination of the two.

The processing unit may be a standard programmable processor that performs arithmetic and logic operations necessary for the operation of the computing device. While only one processing unit is shown, multiple processors may be present. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism (124) for communicating information among various components of the computing device.

Computing devices may have additional features/functionality. For example, the computing device may include additional storage such as removable storage and non-removable storage including, but not limited to, magnetic or optical disks or tapes. Computing devices may also contain network connection(s) that allow the device to communicate with other devices, such as over the communication pathways described herein. The network connection(s) may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards such as code division multiple access (CDMA), global system for mobile communications (GSM), long-term evolution (LTE), worldwide interoperability for microwave access (WiMAX), and/or other air interface protocol radio transceiver cards, and other well-known network devices. Computing devices may also have input device(s) associated with a User Device (126) such as keyboards, keypads, switches, dials, mice, trackballs, touch screens, voice recognizers, card readers, paper tape readers, or other well-known input devices. Output device(s) may also be associated with the User Device (126) such as printers, video monitors, liquid crystal displays (LCDs), touch screen displays, displays, speakers, etc., may also be included. The additional devices may be connected to the bus in order to facilitate the communication of data among the components of the computing device. All these devices are well known in the art and need not be discussed at length here.

The processing unit may be configured to execute program code encoded in tangible, computer-readable media on the memory (110). Tangible, computer-readable media refers to any media that is capable of providing data that causes the computing device (i.e., a machine) to operate in a particular fashion. Various computer-readable media may be utilized to provide instructions to the processing unit for execution. Example tangible, computer-readable media may include but is not limited to volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In light of the above, it should be appreciated that many types of physical transformations take place in the computer architecture to store and execute the software components presented herein. It also should be appreciated that the computer architecture may include other types of computing devices, including hand-held computers, embedded computer systems, personal digital assistants, and other types of computing devices known to those skilled in the art.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language, if desired. In any case, the language may be a compiled or interpreted language, and it may be combined with hardware implementations.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications and publications as listed below and throughout this document are hereby incorporated by reference in their entirety herein.

### REFERENCES

[1] Kanwar M K, Thenappan T, Vachiery J L. Update in treatment options in pulmonary hypertension. J Heart Lung Transplant. 2016; 35(6):695-703.
[2] Benza R L, Miller D P, Barst R J, Badesch D B, Frost A E, McGoon M D. An evaluation of long-term survival from time of diagnosis in pulmonary arterial hypertension from the REVEAL Registry. Chest. 2012; 142(2):448-56.
[3] Galie N, Humbert M, Vachiery J L, Gibbs S, Lang I, Torbicki A, et al. 2015 ESC/ERS Guidelines for the diagnosis and treatment of pulmonary hypertension: The Joint Task Force for the Diagnosis and Treatment of Pulmonary Hypertension of the European Society of Cardiology (ESC) and the European Respiratory Society (ERS): Endorsed by: Association for European Paediatric and Congenital Cardiology (AEPC), International Society for Heart and Lung Transplantation (ISHLT). Eur Respir J. 2015; 46(4):903-75.
[4] Benza R L, Farber H W, Selej M, Gomberg-Maitland M. Assessing risk in pulmonary arterial hypertension: what we know, what we don't. Eur Respir J. 2017; 50(2).
[5] Hoeper M M, Kramer T, Pan Z, Eichstaedt C A, Spiesshoefer J, Benjamin N, et al. Mortality in pulmonary arterial hypertension: prediction by the 2015 European pulmonary hypertension guidelines risk stratification model. Eur Respir J. 2017; 50(2).
[6] D'Alonzo G E, Barst R J, Ayres S M, Bergofsky E H, Brundage B H, Detre K M, et al. Survival in patients with primary pulmonary hypertension. Results from a national prospective registry. Ann Intern Med. 1991; 115(5):343-9.
[7] Boucly A, Weatherald J, Savale L, Jais X, Cottin V, Prevot G, et al. Risk assessment, prognosis and guideline implementation in pulmonary arterial hypertension. Eur Respir J. 2017; 50(2).
[8] Kanwar M K, Gomberg-Maitland M, Hoeper M, Pausch C, Pittow D, Strange G, et al. Risk stratification in pulmonary arterial hypertension using Bayesian analysis. Eur Respir J. 2020.
[9] Samokhin A O, Stephens T, Wertheim B M, Wang R S, Vargas S O, Yung L M, et al. NEDD9 targets COL3A1 to promote endothelial fibrosis and pulmonary arterial hypertension. Sci Transl Med. 2018; 10(445).
[10] Manders E, Rain S, Bogaard H J, Handoko M L, Stienen G J, Vonk-Noordegraaf A, et al. The striated muscles in pulmonary arterial hypertension: adaptations beyond the right ventricle. Eur Respir J. 2015; 46(3):832-42.
[11] Hsu S, Kokkonen-Simon K M, Kirk J A, Kolb T M, Damico R L, Mathai S C, et al. Right Ventricular Myofilament Functional Differences in Humans With Systemic Sclerosis-Associated Versus Idiopathic Pulmonary Arterial Hypertension. Circulation. 2018; 137(22):2360-70.
[12] Kanwar M, Raina A, Lohmueller L, Kraisangka J, Benza R. The Use of Risk Assessment Tools and Prognostic Scores in Managing Patients with Pulmonary Arterial Hypertension. Curr Hypertens Rep. 2019; 21(6):45.
[13] Ford H J, Le Varge B. The Pursuit of Risk Assessment and Stratification Tools in Pulmonary Arterial Hypertension: Have We Seen the Lite? Chest. 2021; 159(1):14-6.
[14] Sahay S, Melendres-Groves L, Pawar L, Cajigas H R, Pulmonary Vascular Diseases Steering Committee of the American College of Chest P. Pulmonary Hypertension Care Center Network: Improving Care and Outcomes in Pulmonary Hypertension. Chest. 2017; 151(4):749-54.
[15] Galie N, Channick R N, Frantz R P, Grunig E, Jing Z C, Moisceva O, et al. Risk stratification and medical therapy of pulmonary arterial hypertension. Eur Respir J. 2019; 53(1).
[16] Rosenzweig E B, Abman S H, Adatia I, Beghetti M, Bonnet D, Haworth S, et al. Paediatric pulmonary arterial hypertension: updates on definition, classification, diagnostics and management. The European respiratory journal. 2019; 53(1):1801916.
[17] Perer A, Wang F, Hu J. Mining and exploring care pathways from electronic medical records with visual analytics. J Biomed Inform. 2015; 56:369-78.
[18] Kraisangka J, Druzdzel M J, Lohmueller L C, Kanwar M K, Antaki J F, Benza R L. Bayesian Network vs. Cox's Proportional Hazard Model of PAH Risk: A Comparison. 2019. Cham. Springer International Publishing: 139-49.
[19] Kraisangka J, Lohmueller L C, Kanwar M K, Zhao C, Druzdzel M J, Antaki J F, et al. Derivation of a Bayesian Network Model from an Existing Risk Score Calculator for Pulmonary Arterial Hypertension. The Journal of Heart and Lung Transplantation. 2019; 38(4):S487-S8.
[20] Kraisangka J, Druzdzel M J. A Bayesian Network Interpretation of the Cox's Proportional Hazard Model. Int J Approx Reason. 2018; 103:195-211.
[21] Scott J V, Kraisangka J, Kanwar M, Druzdzel M, Antaki J, Vizza D, et al. Bayesian Network Modeling: The Future of Pulmonary Arterial Hypertension Risk Stratification Through the PHORA Initiative. B97. WHAT'S NEW IN CLINICAL RESEARCH IN PULMONARY HYPERTENSION: LESSONS FROM THE BEST ABSTRACTS: American Thoracic Society; 2020:A4245-A.
[22] Benza R L, Gomberg-Maitland M, Elliott C G, Farber H W, Foreman A J, Frost A E, et al. Predicting Survival in Patients with Pulmonary Arterial Hypertension: The REVEAL Risk Score Calculator 2.0 and Comparison with ESC/ERS-Based Risk Assessment Strategies. Chest. 2019.
[23] Strange G, Lau E M, Giannoulatou E, Corrigan C, Kotlyar E, Kermeen F, et al. Survival of Idiopathic Pulmonary Arterial Hypertension Patients in the Modern Era in Australia and New Zealand. Heart Lung Circ. 2018; 27(11):1368-75.
[24] D'Agostino R B, Sr., Pencina M J, Massaro J M, Coady S. Cardiovascular Disease Risk Assessment: Insights from Framingham. Glob Heart. 2013; 8(1):11-23.
[25] Scott J V, Kanwar M, Garnett C, Stockbridge N, Benza R L. Pulmonary Arterial Registry Risk Algorithms Improve Upon Clinical Trial Enrichment Methodology. B27. UP-TO-DATE PAH ASSESSMENT AND MANAGEMENT: American Thoracic Society; 2020:A2920-A.
[26] Scott J V, Garnett C E, Kanwar M K, Stockbridge N L, Benza R L. Enrichment Benefits of Risk Algorithms for Pulmonary Arterial Hypertension Clinical Trials. Am J Respir Crit Care Med. 2020.
[27] Scott J V, Carey L, Garnett C G, Kanwar M K, Benza R. Upfront Combination Versus Monotherapy in Pulmonary Arterial Hypertension (PAH): Can Advanced Risk Stratification Tell Us the Real Benefit?; 2019.
[28] Frost A E, Hoeper M M, Barbera J A, Vachiery J L, Blair C, Langley J, et al. Risk-stratified outcomes with initial combination therapy in pulmonary arterial hypertension: Application of the REVEAL risk score. J Heart Lung Transplant. 2018; 37(12):1410-7.
[29] Galie N, Humbert M, Vachiery J L, Gibbs S, Lang I, Torbicki A, et al. [2015 ESC/ERS Guidelines for the diagnosis and treatment of pulmonary hypertension]. Kardiol Pol. 2015; 73(12):1127-206.
[30] Scott J, Lohmueller L, Kraisangka J, Kanwar M, Benza R. HEMODYNAMIC PARAMETERS IN PREDICTING SURVIVAL IN PULMONARY ARTERIAL HYPERTENSION. CHEST. 2019; 156(4):A1173-A4.
[31] Simpson C E, Damico R L, Hassoun P M, Martin L J, Yang J, Nies M K, et al. Noninvasive Prognostic Biomarkers for Left-Sided Heart Failure as Predictors of Survival in Pulmonary Arterial Hypertension. Chest. 2020; 157(6):1606-16.
[32] Simpson C E, Chen J Y, Damico R L, Hassoun P M, Martin L J, Yang J, et al. Cellular sources of interleukin-6 and associations with clinical phenotypes and outcomes in pulmonary arterial hypertension. Eur Respir J. 2020; 55(4).
[33] Benza R L, Gomberg-Maitland M, Demarco T, Frost A E, Torbicki A, Langleben D, et al. ET-1 Pathway Polymorphisms Affect Outcome in Pulmonary Arterial Hypertension. American Journal of Respiratory and Critical Care Medicine. 2015.
[34] Karnes J H, Wiener H W, Schwantes-An T H, Natarajan B, Sweatt A J, Chaturvedi A, et al. Genetic Admixture and Survival in Diverse Populations with Pulmonary Arterial Hypertension. Am J Respir Crit Care Med. 2020; 201(11):1407-15.
[35] Hurdman J, Condliffe R, Elliot C A, Davies C, Hill C, Wild J M, et al. ASPIRE registry: assessing the Spectrum of Pulmonary hypertension Identified at a REferral centre. Eur Respir J. 2012; 39(4):945-55.
[36] Lewis R A, Johns C S, Cogliano M, Capener D, Tubman E, Elliot C A, et al. Identification of Cardiac Magnetic Resonance Imaging Thresholds for Risk Stratification in Pulmonary Arterial Hypertension. Am J Respir Crit Care Med. 2020; 201(4):458-68.
[37] Strange G, Playford D, Stewart S, Deague J A, Nelson H, Kent A, et al. Pulmonary hypertension: prevalence and mortality in the Armadale echocardiography cohort. Heart. 2012; 98(24):1805-11.
[38] Strange G, Celermajer D S, Marwick T, Prior D, Ilton M, Codde J, et al. The National Echocardiography Database Australia (NEDA): Rationale and methodology. Am Heart J. 2018; 204:186-9.
[39] Bycroft C, Freeman C, Petkova D, Band G, Elliott L T, Sharp K, et al. The UK Biobank resource with deep phenotyping and genomic data. Nature. 2018; 562(7726):203-9.
[40] Zhou X. Bayesian Lasso for Detecting Rare Genetic Variants Associated with Common Diseases. 2019.
[41] FHIR.
[42] Tibshirani R. Regression Shrinkage and Selection Via the Lasso. Journal of the Royal Statistical Society: Series B (Methodological). 1996; 58(1):267-88.
[43] Li JFaR. Variable Selection via Nonconcave Penalized Likelihood and its Oracle Properties. Journal of the American Statistical Association. 2001; 96(456):1348-60.
[44] Breiman L. Random Forests. Machine Learning. 2001; 45:5-32.
[45] Druzdel M. GeNIe
[46] Scutari M. Learning Bayesian Networks with the bnlearn R Package. Journal of Statistical Software. 2010; 35(3):1-22.
[47] Menden M., Wang, D., et. al. “Community assessment to advance computational prediction of cancer drug combinations in a pharmacogenomic screen”, Nat. Comm. 10, 2674 (2019).
[48] Ruyssinck, J., Huynh-Thu, V., et al. “NIMEFI: gene regulatory network inference using multiple ensemble feature importance algorithms”, PLOS One. 2014 Mar. 25; 9(3):e92709.
[51] Andrews, B., Ramsey, J., and Cooper, G., “Scoring Bayesian networks of mixed variables”. Int J Data Sci Anal 6, 3-18 (2018).
[52] McGeachie M J, Chang H-H, Weiss ST, “CGBayesNets: Conditional Gaussian Bayesian Network Learning and Inference with Mixed Discrete and Continuous Data”. PLOS Comput Biol 10(6): e1003676 (2014).
[53] Stajduhar, I., and Dalbelo-Basic, B., “Learning Bayesian networks from survival data using weighting censored instances”, J. Biomed. Informatics 2010; 43(4):613-622.
[54] Nir A L A, Rauh M. NT-pro-B-type natriuretic peptide in infants and children: reference values based on combined data from four studies. Pediatr Cardiol. 2009; 30(1):3-8.
[55] https://www.icd10data.com/, Accessed 8 Jun. 2023.
[56] Wang APaF. Frequence: Interactive Mining and Visualization of Temporal Frequent Event Sequences. ACM Conference on Intelligent User Interfaces (IUI 2014); 2014.
[57] Gotz APaD. Data-Driven Exploration of Care Plans for Patients. ACM Conference on Human Factors in Computing Systems (CHI 2013); 2013.
[58] Kylhammar D, Kjellström B. Hjalmarsson C, et al. A comprehensive risk stratification at early follow-up determines prognosis in pulmonary arterial hypertension. Eur Heart J 2018; 39: 4175-4181.
[59] Kraisangka J, Druzdzel M J, Lohmueller L C, et al. Bayesian network vs. Cox's proportional hazard model of PAH risk: a comparison. In: Riaño D, Wilk S, ten Teije A, eds. Artificial Intelligence in Medicine. Cham, Springer International Publishing, 2019.
[60] Friedman N, Geiger D, Goldszmidt M. Bayesian network classifiers. Machine Learning 1997; 29:131-163.
[61] Strange G, Lau E M, Giannoulatou E, et al. Survival of idiopathic pulmonary arterial hypertension patients in the modern era in Australia and New Zealand. Heart Lung Circ 2018; 27:1368-1375.
[62] Kanwar M K, Lohmueller L C, Kormos R L, et al. A Bayesian model to predict survival after left ventricular assist device implantation. JACC Heart Fail 2018; 6:771-779.
[63] Loghmanpour N A, Kormos R L, Kanwar M K, et al. A Bayesian model to predict right ventricular failure following left ventricular assist device therapy. JACC Heart Fail 2016; 4:711-721.
[64] Miranda E, Irwansyah E, Amelga A Y, et al. Detection of cardiovascular disease risk's level for adults using naïve Bayes classifier. Healthc Inform Res 2016; 22:196-205.
[65] Cao K, Xu J, Zhao W Q. Artificial intelligence on diabetic retinopathy diagnosis: an automatic classification method based on grey level co-occurrence matrix and naive Bayesian model. Int J Ophthalmol 2019; 12:1158-1162.
[66] Cox D R. Regression models and life-tables. JR Stat Soc B 1972; 34:187-220.
[67] Hernán M A. The hazards of hazard ratios. Epidemiology 2010; 21:13-15.

### Conclusion

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to the arrangement of steps or operational flow; plain meaning derived from grammatical organization or punctuation; the number or type of embodiments described in the specification.

While the methods and systems have been described in connection with certain embodiments and specific examples, it is not intended that the scope be limited to the particular embodiments set forth, as the embodiments herein are intended in all respects to be illustrative rather than restrictive.

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

The term “compound,” as used herein, is meant to include all stereoisomers, geometric isomers, tautomers, and isotopes of the structures depicted. Compounds herein identified by name or structure as one particular tautomeric form are intended to include other tautomeric forms unless otherwise specified.

Compounds are described using standard nomenclature. Unless defined otherwise, all technical and scientific terms used herein have the same meaning as is commonly understood by one of skill in the art to which this invention belongs.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formally MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

All compounds, and salts thereof, can be found together with other substances such as water and solvents (e.g., hydrates and solvates).

Compounds provided herein also can include tautomeric forms. Tautomeric forms result from the swapping of a single bond with an adjacent double bond together with the concomitant migration of a proton. Tautomeric forms include prototropic tautomers, which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include hydrogen, tritium, and deuterium.

Also provided herein are salts of the compounds described herein. It is understood that the disclosed salts can refer to derivatives of the disclosed compounds wherein the parent compound is modified by converting an existing acid or base moiety to its salt form. Examples of the salts include, but are not limited to, mineral or organic acid salts of basic residues such as amines; alkali or organic salts of acidic residues such as carboxylic acids; and the like. The salts of the compounds provided herein include the conventional non-toxic salts of the parent compound formed, for example, from non-toxic inorganic or organic acids. The salts of the compounds provided herein can be synthesized from the parent compound that contains a basic or acidic moiety by conventional chemical methods. Generally, such salts can be prepared by reacting the free acid or base forms of these compounds with a stoichiometric amount of the appropriate base or acid in water or an organic solvent or in a mixture of the two. In various aspects, nonaqueous media like ether, ethyl acetate, alcohols (e.g., methanol, ethanol, isopropanol, or butanol), or acetonitrile (ACN) can be used.

As used herein, a “monomer” refers to a molecule capable of reacting together with other monomer molecules to form a larger polymer chain or three-dimensional network via polymerization.

As used herein, a “prepolymer” refers to a monomer or system or monomers that have been reacted into a composition having an intermediate-molecular mass state and capable of being further polymerized by reactive groups into a fully cured, high-molecular-mass state. Prepolymers as used herein may refer to mixtures of reactive polymers or mixtures of reactive polymers with unreacted monomers.

As used herein, a “resin” refers to mixture of monomers and/or prepolymers or related substances capable of converting into a rigid polymer by the cross-linking of polymer chains (i.e., curing).

Throughout this application, various publications may have been referenced. The disclosures of these publications in their entireties are hereby incorporated by reference into this application in order to more fully describe the state of the art to which this invention pertains.
[1] M. Meem, S. Banerji, C. Pies, T. Oberbiermann, A. Majumder, B. Sensale-Rodriguez, and R. Menon, Large-Area, High-Numerical-Aperture Multi-Level Diffractive Lens via Inverse Design, Optica 7, 252 (2020).
[2] N. Mohammad, M. Meem, X. Wan, and R. Menon, Full-Color, Large Area, Transmissive Holograms Enabled by Multi-Level Diffractive Optics, Sci. Rep. 7, 5789 (2017).
[3] M. J. Allen, H.-M. Lien, N. Prine, C. Burns, A. K. Rylski, X. Gu, L. M. Cox, F. Mangolini, B. D. Freeman, and Z. A. Page, Multimorphic Materials: Spatially Tailoring Mechanical Properties via Selective Initiation of Interpenetrating Polymer Networks, Adv. Mater. 35, 2210208 (2023).
[4] T. L. Andrew, H.-Y. Tsai, and R. Menon, Confining Light to Deep Subwavelength Dimensions to Enable Optical Nanopatterning, Science (80-.). 324, 917 (2009).
[5] E. S. Rosker, M. T. Barako, E. Nguyen, D. Dimarzio, K. Kisslinger, D. W. Duan, R. Sandhu, M. S. Goorsky, and J. Tice, Approaching the Practical Conductivity Limits of Aerosol Jet Printed Silver, ACS Appl. Mater. Interfaces 12, 29684 (2020).
[6] V. Hahn, P. Rictz, F. Hermann, P. Müller, C. Barner-Kowollik, T. Schlöder, W. Wenzel, E. Blasco, and M. Wegener, Light-Sheet 3D Microprinting via Two-Colour Two-Step Absorption, Nat. Photonics 16, 784 (2022).
[7] T. F. Scott, B. A. Kowalski, A. C. Sullivan, C. N. Bowman, and R. R. McLeod, Two-Color Single-Photon Photoinitiation and Photoinhibition for Subdiffraction Photolithography, Science (80-.). 324, 913 (2009).
[8] K. S. Mason, S.-Y. Huang, S. K. Emslie, Q. Zhang, S. M. Humphrey, J. L. Sessler, and Z. A. Page, 3D-Printed Porous Supramolecular Sorbents for Cobalt Recycling, J. Am. Chem. Soc. 146, 4078 (2024).
[9] L. Shi, B. Li, C. Kim, P. Kellnhofer, and W. Matusik, Towards Real-Time Photorealistic 3D Holography with Deep Neural Networks, Nature 591, 234 (2021).
[10] R. Menon and N. Brimhall, Perspectives on Imaging with Diffractive Flat Optics, ACS Photonics 10, 1046 (2023).
[11] G. Pariani, R. Castagna, R. Menon, C. Bertarelli, and A. Bianco, Modeling Absorbance-Modulation Optical Lithography in Photochromic Films, Opt. Lett. 38, 3024 (2013).
[12] A. Majumder, P. L. Helms, T. L. Andrew, and R. Menon, A Comprehensive Simulation Model of the Performance of Photochromic Films in Absorbance-Modulation-Optical-Lithography, AIP Adv. 6, 35210 (2016).
[13] D. Lin, T. M. Hayward, W. Jia, A. Majumder, B. Sensale-Rodriguez, and R. Menon, Inverse-Designed Multi-Level Diffractive Doublet for Wide Field-of-View Imaging, ACS Photonics 10, 2661 (2023).
[14] W. Jia, D. Lin, R. Menon, and B. Sensale-Rodriguez, Machine Learning Enables the Design of a Bidirectional Focusing Diffractive Lens, Opt. Lett. 48, 2425 (2023).
[15] S. Banerji, A. Majumder, A. Hamrick, R. Menon, and B. Sensale-Rodriguez, Ultra-Compact Integrated Photonic Devices Enabled by Machine Learning and Digital Metamaterials, OSA Contin. 4, 602 (2021).
[16] W. Jia, D. Lin, R. Menon, and B. Sensale-Rodriguez, Multifocal Multilevel Diffractive Lens by Wavelength Multiplexing, Appl. Opt. 62, 6931 (2023).
[17] FRONTERA: The Fastest Academic Supercomputer in the U.S., (unpublished).
[18] Top500.org, TOP500 LIST—June 2024, (unpublished).
[19] Y. Zhao, C.-H. Chang, R. K. Heilmann, and M. L. Schattenburg, Phase Control in Multiexposure Spatial Frequency Multiplication, J. Vac. Sci. Technol. B Microelectron. Nanom. Struct. 25, 2439 (2007).
[20] E. E. Moon, L. Chen, P. N. Everett, M. K. Mondol, and H. I. Smith, Interferometric-Spatial-Phase Imaging for Six-Axis Mask Control, J. Vac. Sci. Technol. B Microelectron. Nanom. Struct. 21, 3112 (2003).
[21] Q. Yang, X. A. Zhang, A. Bagal, W. Guo, and C.-H. Chang, Antireflection Effects at Nanostructured Material Interfaces and the Suppression of Thin-Film Interference, Nanotechnology 24, 235202 (2013).

What is claimed is:

1. A clinical decision support system comprising:
a processor;
a memory having instructions stored thereon; and
a means for input and output, wherein at least one set of input variable data are provided by the input means,
wherein execution of the instructions by the processor causes the processor to execute a risk algorithm configured to generate a risk score value for a disease area, and
wherein the clinical decision support system is configured to display a set of risk score value (e.g., in a plotted line, the measured metrics of the patient) computed by the one or more risk algorithms associated with a first set of input variable data.

2. The clinical decision support system of claim 1, wherein the clinical decision support system is configured to display a second risk score value (e.g., in the same plotted line, the predictive risk assessment) associated with a second set of input variable data or parameters with the displayed first risk score value.

3. The clinical decision support system of claim 2, wherein the first and/or second risk score value is categorized into low risk (>95% chance of survival in 1 year), medium risk (%95-%90 chance of survival in 1 year), high risk (<90% chance of survival in 1 year).

4. The clinical decision support system of claim 1, wherein execution of the instructions by the processor causes the processor to query a lookup table of clinical treatment guidelines for the disease area.

5. The clinical decision support system of claim 1, wherein the memory further comprises a database for storing input variable data for one or more input instances.

6. The clinical decision support system of claim 1, wherein execution of the instructions by the processor causes the processor to calculate the influence of the set of input variable data on the associated risk score value.

7. The clinical decision support system of claim 1, wherein one of the risk algorithm comprises an ensemble of one or more Bayesian (neural) networks, wherein the one or more Bayesian networks are tree-augmented Naive Bayes (TAN) networks.

8. The clinical decision support system of claim 1, wherein the risk algorithm is one of a plurality of risk algorithms, each associated with a different disease area.

9. The clinical decision support system of claim 1, wherein the disease area is Pulmonary Arterial Hypertension.

10. A clinical decision support system comprising:
a processor;
a memory having instructions stored thereon; and
a means for input and output, wherein at least one set of input variable data are provided by the input means,
wherein execution of the instructions by the processor causes the processor to execute one or more risk algorithms, wherein each of the one or more risk algorithms is configured to generate a risk score value for a disease area associated with the risk algorithm using a subset of the input variable data.

11. The clinical decision support system of claim 10, wherein the disease area is Pulmonary Arterial Hypertension.
